SEQUENCE LISTING (8 sequences)

SEQ ID NO: 1 - DNA, 20 bp, Artificial Sequence
  gRNA1
aggccacatagtgctgcaca

SEQ ID NO: 2 - DNA, 20 bp, Artificial Sequence
  gRNA2
tgtacagcagtggctggtgg

SEQ ID NO: 3 - DNA, 18 bp, Artificial Sequence
  gRNA1 TGFBR2
cggctgaggagcggaaga

SEQ ID NO: 4 - DNA, 18 bp, Artificial Sequence
  gRNA2 TGFBR2
tggaggtgagcaatcccc

SEQ ID NO: 5 - DNA, 20 bp, Artificial Sequence
  T7 promoter
ttaatacgactcactatagg

SEQ ID NO: 6 - DNA, 20 bp, Artificial Sequence
  Target sequence
gttttagagctagaaatagc

SEQ ID NO: 7 - DNA, 20 bp, Artificial Sequence
  Exon 4 forward primer
cgtctggactccaactgctt

SEQ ID NO: 8 - DNA, 20 bp, Artificial Sequence
  Exon 4 Reverse Primer
gtacaaagggctgcaccagt

(19) United States
(12) Patent Application Publication
(10) Pub. No.: US 2021/0230548 A1
(43) Pub. Date: Jul. 29, 2021

(54) NATURAL KILLER CELLS ENGINEERED TO EXPRESS CHIMERIC ANTIGEN RECEPTORS WITH IMMUNE CHECKPOINT BLOCKADE

(71) Applicant: Board of Regents, The University of Texas System, Austin, TX (US)

(72) Inventors: May Daher, Houston, TX (US); Rafet Basar, Houston, TX (US); Elizabeth Shpall, Houston, TX (US); Katy Rezvani, Houston, TX (US)

(21) Appl. No.: 17/050,775

(22) PCT Filed: May 3, 2019
(86) PCT No.: PCT/US2019/030721

§ 371 Date: Oct. 26, 2020

Related U.S. Application Data
(60) Provisional application No. 62/666,965, filed on May 4, 2018.
(60) Provisional application No. 62/666,665, filed on May 3, 2018.

(51) Int. Cl. C12N 5/0783; C07K 14/725; C07K 14/54; C07K 16/28; A61K 35/17; C07K 14/47; A61P 35/00

(52) U.S. Cl. CPC C12N 5/0646; C07K 14/7051; C07K 14/5443; C07K 16/2803; C07K 14/5434; C07K 14/54; C07K 2319/33; C07K 14/4703; A61P 35/00; C12N 2510/00; C12N 2310/20; C12N 2800/80; A61K 35/17

(57) ABSTRACT

Provided herein are methods for producing NK cells expressing chimeric antigen receptors and having no expression of CISH. Further provided are methods for treating diseases by administering the CAR NK cells.

This application claims the benefit of United States Provisional patent Application Nos. 62/666,665, filed May 3, 2018, and 62/666,965, filed May 4, 2018, which are both incorporated herein by reference in their entirety.

### INCORPORATION OF SEQUENCE LISTING

The sequence listing that is contained in the file named “UTFC1366WO.txt”, which is 2 KB (as measured in Microsoft Windows) and was created on May 3, 2019, is filed herewith by electronic submission and is incorporated by reference herein.

### BACKGROUND

### 1. Field

The present invention relates generally to the fields of immunology and medicine. More particularly, it concerns natural killer (NK) cells engineered to express a chimeric antigen receptor with disrupted expression of an immune checkpoint gene.

### 2. Description of Related Art

In recent years, adoptive cellular therapy using autologous T cells transduced with chimeric antigen receptor (CAR) has proven to be a very powerful approach for the treatment of cancer, leading to Food and Drug Administration (FDA) approvals for B cell leukemia and lymphoma. However, challenges remain, including uncoupling cytotoxicity against tumor cells from systemic toxicity, finding solutions for target antigen negative relapses, and developing universal off-the-shelf cell therapy products to avoid the logistic hurdles of generating autologous products, while managing the challenges of allogeneic T cell products, such as graft-versus-host disease (GVHD) (Hartmann et al., 2017).

Natural killer (NK) cells are attractive contenders since they mediate effective cytotoxicity against tumor cells and unlike T cells, lack the potential to cause GVHD in the allogeneic setting. Thus, NK cells could be made available as an off-the-shelf cellular therapy product for immediate clinical use (Daher et al., 2018). Cord blood (CB) is a readily available source of allogeneic NK cells with the potential for widespread clinical scalability. The potent activity of CAR transduced NK cells has been proven in the preclinical setting in different tumor models, and clinical trials of allogeneic CAR-transduced NK cells are currently underway (Mehta et al., 2018). While immune checkpoint molecules, such as PD-1, are being targeted to enhance the function of CAR-T cells, no immune checkpoint molecule has been targeted in CAR-NK cells to date (Gay et al., 2017).

Functional NK cells can be derived from several sources. Autologous NK cells can be reproducibly generated in vitro, but have limited activity against autologous tumors, which may not be overcome by CAR engineering. Cord blood (CB) is a readily available source of allogeneic NK cells with clear advantages. CB is available as an off-the-shelf frozen product, an advantage that has been bolstered by methods to generate large numbers of highly functional NK cells from frozen CB units ex vivo. The generation of CAR-transduced NK cells from frozen CB units stored in large global CB bank inventories holds promise for widespread scalability that cannot be replicated with individual adult donors who require screening and leukapheresis. However, a major disadvantage of NK cells is their lack of persistence after adoptive transfer in the absence of cytokine support. Therefore, engineering CAR-NK cells to express cytokines to enhance their persistence is important for their clinical activity. By doing so, CAR-NK cells can upregulate checkpoint molecules which could limit their functionality. Therefore, there is a need to delete checkpoint molecules in CAR engineered NK cells to enhance their potency and clinical activity.

### SUMMARY

In a first embodiment, the present disclosure provides isolated natural killer (NK) cells engineered to express (1) a chimeric antigen receptor (CAR) and/or a T cell receptor (TCR) and (2) human IL-15 (hIL-15) and to have essentially no expression of CISH. In some aspects, the NK cell is engineered to express a CAR. In certain aspects, the NK cell is engineered to express a TCR. In particular aspects, the NK cell is engineered to express a CAR and TCR or 3 or 4 antigen receptors. In some aspects, the NK cells are GMP-compliant. In certain aspects, the NK cells are allogeneic or autologous.

In some aspects, the NK cell is derived from cord blood, peripheral blood, bone marrow, CD34⁺ cells, or iPSCs. In certain aspects, the NK cell is derived from cord blood. In particular aspects, the cord blood has previously been frozen.

In certain aspects, the CAR and/or TCR has antigenic specificity for CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD33, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2.

In additional aspects, the NK cell further expresses a second or third cytokine. In particular aspects, the cytokine is IL-15, IL-21 or IL-12.

In another embodiment, there is provided a method for producing NK cells of the embodiments comprising obtaining a starting population of NK cells; culturing the starting population of NK cells in the presence of artificial presenting cells (APCs); introducing a CAR and/or TCR expression vector into the NK cells; expanding the NK cells in the presence of APCs, thereby obtaining expanded NK cells; and disrupting the expression of CISH in the expanded NK cells.

In some aspects, disrupting expression comprises using CRISPR-mediated gene silencing. In certain aspects, CRISPR-mediated gene silencing comprises contacting the CAR NK cells with sgRNA and Cas9. In particular aspects, the sgRNA targets exon 4 of CISH. In specific aspects, the sgRNA comprises crRNA1 which has the sequence AGGCCACATAGTGCTGCACA (SEQ ID NO:1) and crRNA2 which has the sequence TGTACAGCAGTGGCTGGTGG (SEQ ID NO:2).

In some aspects, the starting population of NK cells is obtained by isolating mononuclear cells using a ficoll-paque density gradient. In particular aspects, the APCs are gamma-irradiated APCs. In some aspects, the APCs are universal APCs (uAPCs). In specific aspects, the APCs are engineered to express 41BB and IL-21. In some aspects, the uAPCs are engineered to express (1) CD48 and/or CS1 (CD319), (2) membrane-bound interleukin-21 (mbIL-21), and (3) 41BB ligand (41BBL). In particular aspects, the NK cells and APCs are present at a 1:1 to 1:10 ratio, such as a 1:1, 1:2, 1:3, 1:4, 1:5, 1:6, 1:7, 1:8, 1:9, or 1:10 ratio. In certain aspects, the NK cells and APCs are present at a 1:2 ratio.

In certain aspects, the NK cells in culture with APCs are expanded further in the presence of IL-2. In certain aspects, the IL-2 is present at a concentration of 100-300 U/mL, such as 100, 125, 150, 175, 200, 225, 250, 275, or 300 U/mL. In some aspects, IL-2 is present at a concentration of 200 U/mL.

In some aspects, introducing comprises transduction. In certain aspects, the CAR and/or TCR expression construct is a lentiviral vector or retroviral vector.

In particular aspects, the CAR and/or TCR has antigenic specificity for CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2.

In some aspects, the CAR and/or TCR expression construct further expresses a cytokine, such as 2 or 3 cytokines. In some aspects, the cytokine is IL-15, IL-21, or IL-12.

In some aspects, the NK cell has activated mammalian target of rapamycin (mTOR) signaling as compared to an NK cells with CISH expression. In certain aspects, the NK cell has increased JAK/STAT signaling as compared to an NK cell with CISH expression.

In additional aspects, the method further comprises cryopreserving the population of expanded NK cells.

Further provided herein is a pharmaceutical composition comprising a population of NK cells of the embodiments or NK cells produced by the method of the embodiments and a pharmaceutically acceptable carrier. In another embodiment, there is provided a composition comprising an effective amount of NK cells of the embodiments or NK cells produced by the method of the embodiments for use in the treatment of a disease or disorder in a subject.

In another embodiment, there is provided the use of a composition comprising an effective amount of NK cells of the embodiments or NK cells produced by the method of the embodiments for the treatment of an immune-related disorder in a subject.

A further embodiment provides a method of treating an immune-related disorder in a subject comprising administering an effective amount of NK cells of the embodiments or NK cells produced by the method of the embodiments to the subject.

In some aspects of the above embodiments, the immune-related disorder is a cancer, autoimmune disorder, graft versus host disease, allograft rejection, or inflammatory condition. In certain aspects, the immune-related disorder is an inflammatory condition and the immune cells have essentially no expression of glucocorticoid receptor. In certain aspects, the subject has been or is being administered a steroid therapy. In some aspects, the NK cells are autologous or allogeneic. In certain aspects, the immune-related disorder is a cancer. In some aspects, the cancer is a solid cancer or a hematologic malignancy.

In additional aspects of the above embodiments, the method further comprises administering at least a second therapeutic agent. In some aspects, the at least a second therapeutic agent comprises chemotherapy, immunotherapy, surgery, radiotherapy, or biotherapy. In some aspects, the NK cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

In some aspects, the NK cells with essentially no expression of CISH have enhanced function as compared to NK cells with expression of CISH. In certain aspects, the enhanced function is measured by intracellular staining for IFN-γ and TNF-α, CD107a degranulation, and tumor killing by 51Cr release assay. In some aspects, the enhanced function is measured by increased expression of granzyme-b, perforin, TRAIL, CD3z, Eomes, T-bet, DAP12, DNAM, CD25 and/or Ki67.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: CIS is upregulated in NK CAR19/IL-15 and correlates with decreased function in a time dependent manner. (FIG. 1A) qPCR for CISH mRNA was done on day 0 (baseline resting NK cells) and on day 7 and day 14 following CAR transduction. 18S was used as housekeeping gene. CISH mRNA is upregulated in a time dependent manner following NKCAR19/IL-15 expansion. (FIG. 1B) Chromium release assay was done to evaluate cytotoxicity or NK CAR19/IL-15 on day 7 and day 14 following CAR transduction. Results show decreased cytotoxicity with time which correlates with upregulation of CISH.

FIGS. 2A-2C: Efficient CISH KO using CRISPR-Cas to target CISH exon 4. CRISPR-Cas9 was used to target CISH exon 4 using 2 gRNAs on day 14 following NK expansion. Knockout efficiency was assessed by (FIG. 2A) PCR for CISH gene on day 3 following KO, (FIG. 2B) western blot for CIS protein on day 7 (FIG. 2C) flow cytometry on day 7.

FIGS. 3A-3D: CISH KO and CAR19/IL-15 transduction improve function and cytotoxicity synergistically. On day 14 following CAR transduction (FIGS. 3A, B) functional studies were performed showing increased cytokine production and degranulation in the CISH KO NT and CISH KO CAR19/IL-15 compared to the WT counterparts. (FIGS. 3C, D) Chromium release assay showing improved cytotoxicity in the CISH KO groups.

FIGS. 4A-4D: CISH KO in NT NK cells and NKCAR19/IL-15 enhances the immune synapse formation with Raji Lymphoma cells. On day 14 following CAR transduction confocal microscopy was performed to compare the immune synapse formation against Raji cells of CISH KO NT and CAR19/IL-15 vs cas9 controls (FIG. 4A) F-actin and CAR accumulation at the synapse is increased (FIG. 4B) MTOC to synapse distance is shortened (FIG. 4C) CAR with F-actin colocalization is increased at the immune synapse (FIG. 4D) trend for increase in CD19, F-actin and CAR colocalization at the immune synapse.

FIGS. 5A-5B: CISH KO CAR19/IL-15 NK cells improve tumor control and survival in a Raji lymphoma mouse model. On day 0, mice were injected with Raji cells (20K/mouse) alone in group 1 or in combination with the different NKCAR19/IL-15 preparations (10⁷ cells/mouse WT, Cas9 control or CISH KO). (FIG. 5A) BLI showed improved tumor control in the group that received the CISH KO cells. (FIG. 5B) Survival curve showing increased survival in the group that received the CISH KO cells.

FIG. 6: At the time of sacrifice, flow cytometry analysis was performed on processed blood, spleen, liver, BM to evaluate presence of Raji and NK CAR. In the mouse that received CISH KO CAR19/IL-15 cells, there was no evidence of Raji lymphoma and a clear population of CAR expressing NK cells was detected in PB and organs.

FIGS. 7A-7E: Efficient CISH KO using CRISPR-Cas9 to target CISH exon 4. (FIG. 7A) RTqPCR results showing time dependent increase in CISH transcription on days 14 and day 21 in both NT and iC9/CAR19/IL-15 transduced NK cells. (FIG. 7B) schematic representation of CRISPR mediated CISH KO using two different guide RNA's (gRNA) (SEQ ID NOs:1-2) targeting exon 4. (FIG. 7C) PCR results done 2 days following RNP electroporation, showing CISH KO efficiency with >90% indel. (FIG. 7D) Western blot done 7 days post RNP electroporation showing percent protein loss, β-actin was used as a reference gene. (FIG. 7E) Sanger sequencing results showing multiple peaks in the NT CISH KO and CAR CISH KO samples compared to their respective cas9 mock controls, which corresponds to CRISPR mediated events of NHEJ. Arrows indicate the base pair position where the gene editing starts.

FIGS. 8A-8F: CISH KO improves function and cytotoxicity in NT and iC9/CAR19/IL-15 transduced NK cells. Functional studies comparing the activity of different NK cell conditions (NT, NT CISH KO, iC9/CAR19/IL-15, iC9/CAR19/Il-15 CISH KO) against Raji lymphoma (FIG. 8A) Representative FACS plots of IFNγ, TNFα and CD107a expression. (FIG. 8B) Bar charts of IFNγ, TNFα and CD107a percent expression in NK cells, arrowbars represent SD (n=6). (FIG. 8C) Chromium release assay at different E:T ratios (5:1, 2:1, 1:1, 0.5:1) (n=3). (FIG. 8D) confocal microscopy showing immune synapse formation between NK cells and Raji lymphoma cells (n=3). (FIG. 8E) plot of MTOC to synapse distance. (FIG. 8F) Incucyte experiment showing percent killing of Raji lymphoma over 12 hrs at different E:T ration 1:1, 1:2 and 1:4, comparing iC9/CAR19/IL-15 vs iC9/CAR19/Il-15 CISH KO.

FIGS. 9A-9H: Phenotype and Molecular signature of CISH KO iC9/CAR19/Il-15 NK cells. (FIG. 9A) Comparative heatmap of mass cytometry data displaying the median of marker expression in CISH KO iC9/CAR19/IL-15 compared to Cas9 control iC9/CAR19/IL-15 (n=2) showing lower or higher expression in CISH KO samples. (FIG. 9B) Representative Visne plots of selected markers showing increased expression in CISH KO iC9/CAR19/IL-15 compared to Cas9 control iC9/CAR19/IL-15. Insert tsne-1 vs tsne2. (FIG. 9C) heatmap showing differentially expressed genes in iC9/CAR19/IL15 NK cells CTRL vs CISH KO (n=2). (FIG. 9D) Gene set enrichment analysis plots showing enrichment in TNFα signaling via NFKB and IFNγ response in CISH KO iC9/CAR19/IL15 NK cells compared to Cas9 control iC9/CAR19/IL15 NK cells. (FIG. 9E) Volcano plot showing some of the genes that are upregulated in CISH KO iC9/CAR19/IL15 NK cells compared to Cas9 control iC9/CAR19/IL-15 NK cells (n=2). (FIG. 9F) Gene set enrichment analysis plots showing enrichment in IL-2/STAT5 signaling, STAT3/IL-6 signaling and inflammatory response in CISH KO iC9/CAR19/IL15 NK cells compared to Cas9 control iC9/CAR19/IL15 NK cells. (FIG. 9G) Representative histograms showing enhanced upregulation of p-STAT5, p-STAT3 and p-PLCg1 in CISH KO iC9/CAR19/IL15 NK cells compared to Cas9 control iC9/CAR19/IL15 NK cells after co-culture with Raji lymphoma cells for 30 min. (FIG. 9H) bar charts showing MFI of p-STAT5, p-STAT3 and p-PLCg1 in Cas9 control vs CISH KO iC9/CAR19/IL15 NK cells.

FIGS. 10A-10L: CISH KO reprograms the metabolism of iC9/CAR19/IL-15 NK cells. (FIG. 10A) GSEA analysis showing enrichment in MTORC1, glycolysis, hypoxia gene sets. (FIG. 10B) Heat map showing differential expression of genes associated with the different enriched metabolic pathways. (FIG. 10C) Network analysis showing upregulated genes (circles) associated with enriched metabolic pathways (nodes): IL-2/STAT5 signaling (brown), mTORC1, Glycolysis and hypoxia, edges between pathway and specific genes are shown. (FIG. 10D) Schematic diagram representing hypothesis of how CIS deletion modulates the metabolic pathway of iC9/CAR19/IL-15 through upregulating MTORC1, HIF-1a and glycolysis. CISH KO modulates the metabolic fitness of NK cells in response to Raji tumor cells. (FIGS. 10E-10F) Sea horse assay showing results of ECAR for NT, NTKO, CAR, CARKO following co-culture with Raji for 2 hrs. (FIG. 10G) Results of glucose colorimetric test done on supernatant collected from wells where different NK cell conditions were co-cultured with Raji for 4 hrs. (FIG. 10H) Glycolysis pathway generated via IPA showing upregulation of glycolysis enzymes represented by the nodes colored in red. (FIG. 10I) Mean fluorescent intensity (MFI) of phospho-S6 in CAR and CAR CISH KO. (FIG. 10J) sea horse assay showing OCR results for NT, NTKO, CAR, CARKO. (FIGS. 10K-10L) Confocal microscopy results evaluating lysosomes, mitochondria and nucleus and statistically comparing their numbers and volumes between NTcas9 and NTKO, CAR cas9 and CAR KO.

FIGS. 11A-11I: CISH KO iC9/CAR19/IL-15 NK cells improve tumor control and survival in a Raji lymphoma mouse model even at low infusion doses. (FIG. 11A) Schematic diagram representing the timeline of the in-vivo experiments. (FIG. 11B) Bioluminescent imaging (BLI) results and (FIG. 11C) survival curves comparing the groups of mice that received Raji alone (n=5) vs Raji+NT (n=5) vs Raji+NT cas9 CTRL (n=5) vs Raji+NT CISH KO (n=5). (FIG. 11D) Percent NK cells out of CD45+ cells present in peripheral blood of mice comparing iC9/CAR19/IL-15 NK cells Cas9 controls vs CISH KO on different days. (FIG. 11E) BLI imaging results (FIG. 11F) average radiance (FIG. 11G) survival curves and (FIG. 11H) body weight curves comparing the following groups of mice Raji alone (n=5) vs Raji+iC9/CAR19/IL-15 3×10e6 cas9 control (n=5) vs iC9/CAR19/IL-15 3×10e6 CISH KO (n=5) vs iC9/CAR19/IL-15 10×10e6 cas9 control (n=5) vs iC9/CAR19/IL-15 10×10e6 CISH KO (n=3). h, representative pathology pictures from liver (left panels) and bone marrow (right panels) showing H&E stain in upper panels and CD20 immunohistochemical staining in lower panels comparing Raji alone vs CAR19/IL-15 CTRL (10×10e6) vs CAR19/IL-15 CISH KO (10×10e6) groups. (FIG. 11I) plots of mice body weights over time among the different groups

FIGS. 12A-12D: Identification of Cas9 off-target sites by GUIDE-Seq and quantification of potential Cas9 off-target cleavage sites using rhAmpSeg™ technology. (FIG. 12A) Sequences of off-target sites identified by GUIDE-Seq for two guides targeting the CISH locus. The guide sequence is listed on top with off-target sites shown below. The on-target site is identified with a black square. Mismatches to the guide are shown and highlighted in with insertions shown in grey. The number of GUIDE-Seq sequencing reads are shown to the right of each site. 10 μM Alt-R crRNA XT complexed to Alt-R tracrRNA was delivered into HEK293 cells that constitutively express Cas9 nuclease by nucleofection. (FIG. 12B) Pie charts indicate the fractional percentage of the total unique, CRISPR-Cas9 specific read counts that are on-target and off-target. Total editing at the on- and off-target sites identified by GUIDE-Seq was measured using rhAmpSeq, a multiplexed targeted enrichment approach for NGS. For each of the two CISH targeting guides amplicons were designed around each Cas9 cleavage site with reads >1% of the on target GUIDE-Seq reads. RNP complexes formed with either WT Cas9 or Alt-R HiFi Cas9 were delivered via electroporation into expanded NK cells. (FIG. 12C) INDEL formation at each targeted loci for CISH guide 1 (panel 1, 11-plex) and CISH guide 2 (panel 2, 70-plex) when a single RNP complex was delivered. (FIG. 12D) INDEL formation at each targeted loci when CISH guide 1 and CISH guide 2 were co-delivered. The on-target locus is indicated with an asterisk

FIGS. 13A-13C: iC9/CAR19/IL-15 transduction and CISH KO efficiency are stable over time. (FIG. 13A) Flow cytometry analysis of CD56 in transduced and control cells. (FIG. 13B) Cell viability of transduced cells. (FIG. 13C) Western blots of various days during transductions.

FIGS. 14A-14D: Phenotype and molecular signature of CISH KO NT NK cells. (FIGS. 14A-14B) Heatmap of gene signature. (FIGS. 14C-14D) Analysis of CISH KO NT NK cells.

FIG. 15: Single dose of CAR19/IL-15 prolongs survival in a Raji lymphoma mouse model but does not lead to cures.

FIGS. 16A-16C: CISH KO iC9/CAR19/IL-15 NK cells can still be eliminated using dimerizer. (FIGS. 16A-16B) Flow cytometry analysis of CISH KO NT NK cells. (FIG. 16C) Percent CAR+ NK cells in blood, liver, spleen, or bone marrow.

FIG. 17: No evidence of Raji lymphoma in mice receiving high dose CISH KO iC9/CAR19/IL-15 NK cells

### DESCRIPTION OF ILLUSTRATIVE EMBODIMENTS

The suppressor of cytokine signaling (SOCS) family of proteins plays an important role in NK cell biology by attenuating cytokine signaling, functional activity, and immunity of NK cells to cancer. One of its members, the cytokine-inducible SH2-containing protein (CIS), encoded by the CISH gene, has been identified as an important intracellular checkpoint molecule in NK cells and is induced by cytokines including IL-15. Thus, the inventors sought to determine if engineered NK cells are subject to the same counter regulatory circuits that physiologically downregulate cytokine signaling in unmodified NK cells, by evaluating the expression of CIS and the consequences of CISH deletion in CAR19/IL-15 transduced CB-NK cells.

The present studies demonstrated a novel approach to combine CAR engineering with CISH knock out (KO) which results in superior tumor control compared to either approach alone. CISH KO enhanced the activity of iC9/CAR19/IL-15-transduced CB-NK cells by increasing JAK/STAT signaling. Moreover, it was shown that targeting CISH modulates the metabolism of CAR-NK cells and enhances their ‘fitness’ by activating the mammalian target of rapamycin (mTOR) signaling pathway and inducing a glycolytic switch in their metabolism. This is the first demonstration that gene editing of an immune checkpoint molecule enhances the activity and fitness of CAR.NK cells by modulating their metabolic pathway.

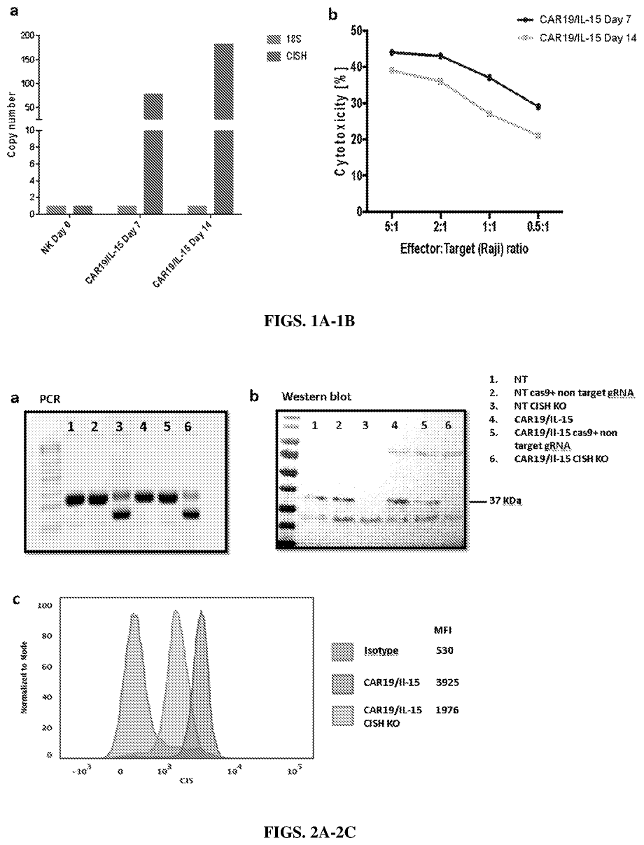

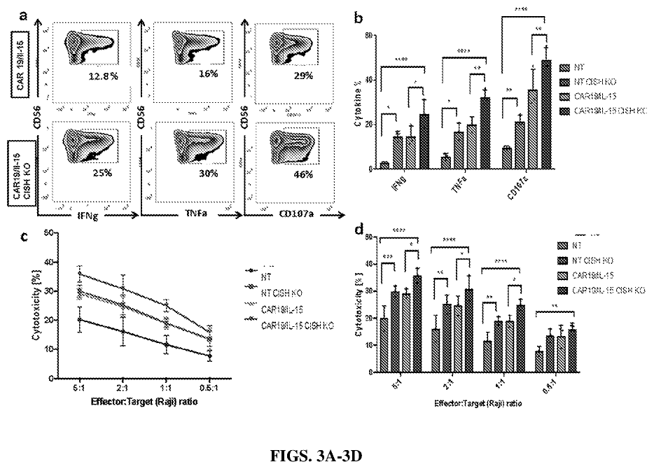

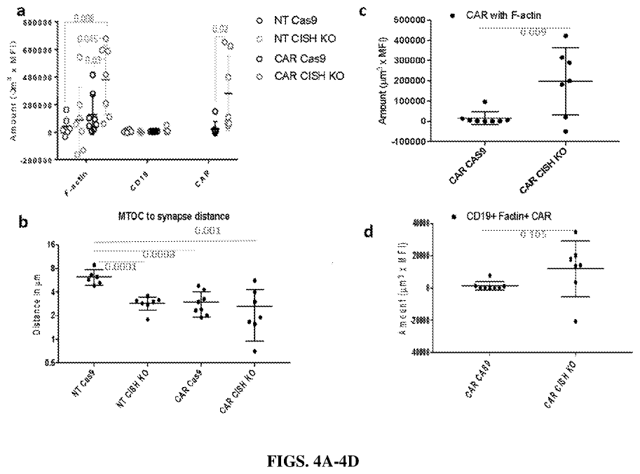

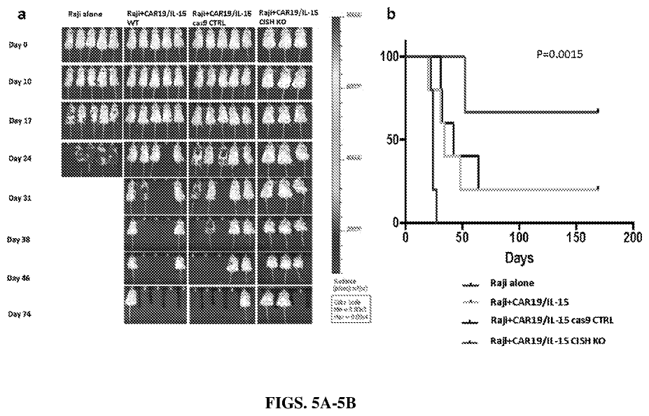

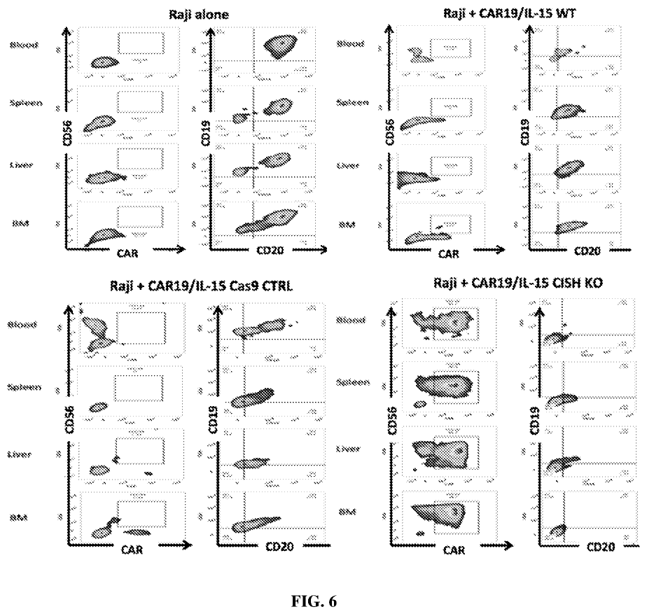

Specifically, the present studies showed that CISH is induced in CAR19/IL-15 transduced CB-NK cells after 7 days of expansion, as determined by mRNA qPCR (FIG. 1). To examine the functional consequences of CISH deletion in CAR.19/IL-15 transduced NK cells, a protocol was developed for combined Cas9 ribonucleoprotein (Cas9 RNP)-mediated gene editing to silence CISH and retroviral transduction with the iC9/CAR.CD19/IL-15 construct. Seven days after transduction of CB-NK cells with the iC9/CAR.19/IL-15 construct, the transduced NK cells were nucleofected with Cas9 alone (Cas9 control) or Cas9 pre-loaded with chemically synthesized crRNA:tracrRNA duplex targeting CISH exon 4. The cells were then cultured with clone 9.mbIL21 and IL-2 (100 iU/ml) for an additional 7 days to enhance expansion. The gene editing efficiency was quantified by PCR (day 2) and the reduction in protein expression levels was determined by flow cytometry (day 7) in CAR-NK cells. CISH knockout resulted in significantly enhanced function of iC9/CAR19/IL-15 transduced CB-NK cells against Raji tumor cells, especially at lower effector:target ratios, as assessed by intracellular staining for IFN-γ and TNF-α, CD107a degranulation, and tumor killing by ⁵¹Cr release assay. Given the promising in vitro data, the engineered CB derived CAR-NK cells were tested in vivo in a murine mouse model of Raji lymphoma. The mice were treated with only one injection of 3 million or 10 million NK cells and it was shown that CISH KO in iC9/CAR19/IL-15 transduced CB-NK enhanced their persistence in vivo and improved survival of the mice at both dose levels but more impressive long term survival was seen with the 10 million dose level.

Accordingly, in certain embodiments, the present disclosure provides methods of producing NK cells by engineering them to express a CAR, such as by retroviral transduction, and disrupting expression of CISH, such as by using CRISPR-Cas9-mediated knockdown of CISH. The CAR construct may express a cytokine, such as IL-15.

The NK cells provided herein can be used to improve adoptive CAR cellular therapies by enhancing the potency, thus enabling an infusion of a lower number of CAR NK cells to patients with decreased toxicity. Thus, further embodiments provide methods of administering adoptive cellular therapy with CAR NK cells to treat cancer patients with a hematologic malignancy, solid cancers, infectious disease, or immune diseases including but not restricted to graft versus host disease.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

An “immune disorder,” “immune-related disorder,” or “immune-mediated disorder” refers to a disorder in which the immune response plays a key role in the development or progression of the disease Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “haplotyping or tissue typing” refers to a method used to identify the haplotype or tissue types of a subject, for example by determining which HLA locus (or loci) is expressed on the lymphocytes of a particular subject. The HLA genes are located in the major histocompatibility complex (MHC), a region on the short arm of chromosome 6, and are involved in cell-cell interaction, immune response, organ transplantation, development of cancer, and susceptibility to disease. There are six genetic loci important in transplantation, designated HLA-A, HLA-B, HLA-C, and HLA-DR, HLA-DP and HLA-DQ. At each locus, there can be any of several different alleles.

A widely used method for haplotyping uses the polymerase chain reaction (PCR) to compare the DNA of the subject, with known segments of the genes encoding MHC antigens. The variability of these regions of the genes determines the tissue type or haplotype of the subject. Serologic methods are also used to detect serologically defined antigens on the surfaces of cells. HLA-A, —B, and -C determinants can be measured by known serologic techniques. Briefly, lymphocytes from the subject (isolated from fresh peripheral blood) are incubated with antisera that recognize all known HLA antigens. The cells are spread in a tray with microscopic wells containing various kinds of antisera. The cells are incubated for 30 minutes, followed by an additional 60-minute complement incubation. If the lymphocytes have on their surfaces antigens recognized by the antibodies in the antiserum, the lymphocytes are lysed. A dye can be added to show changes in the permeability of the cell membrane and cell death. The pattern of cells destroyed by lysis indicates the degree of histologic incompatibility. If, for example, the lymphocytes from a person being tested for HLA-A3 are destroyed in a well containing antisera for HLA-A3, the test is positive for this antigen group.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B-cells, endothelial cells, activated T-cells, and dendritic cells, or molecules, naturally occurring or synthetic capable of presenting antigen, such as purified MHC Class I molecules complexed to ÿ2-microglobulin.

### II. NK CELLS

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, or stem cells. In particular embodiments, the immune cells are isolated from pooled CB. The CB may be pooled from 2, 3, 4, 5, 6, 7, 8, 10, or more units. The immune cells may be autologous or allogeneic. The isolated NK cells may be haplotype matched for the subject to be administered the cell therapy. NK cells can be detected by specific surface markers, such as CD16, CD56, and CD8 in humans with absence of CD3 expression.

In certain aspects, the starting population of NK cells is obtained by isolating mononuclear cells using ficoll density gradient centrifugation. The cell culture may be depleted of any cells expressing CD3, CD14, and/or CD19 cells and may be characterized to determine the percentage of CD56⁺/CD3⁻ cells or NK cells.

The cells are expanded in the presence of the present APCs, such as UAPCs. The expansion may be for about 2-30 days, such as 3-20 days, particularly 12-16 days, such as 12, 13, 14, 15, 16, 17, 18, or 19 days, specifically about 14 days. The NK cells and APCS may be present at a ratio of about 3:1-1:3, such as 2:1, 1:1, 1:2, specifically about 1:2. The expansion culture may further comprise cytokines to promote expansion, such as IL-2, IL-21, and/or IL-18. The cytokines may be present at a concentration of about 10-500 U/mL, such as 100-300 U/mL, particularly about 200 U/mL. The cytokines may be replenished in the expansion culture, such as every 2-3 days. The APCs may be added to the culture at least a second time, such as after CAR transduction.

Following expansion, the immune cells may be immediately infused or may be stored, such as by cryopreservation. In certain aspects, the cells may be propagated for days, weeks, or months ex vivo as a bulk population within about 1, 2, 3, 4, 5 days.

Expanded NK cells can secrete type I cytokines, such as interferon-γ, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

A. CISH

Cytokine-inducible SH2-containing protein (CIS) is encoded by the CISH gene. It is a member of the SOCS family and is an intracellular checkpoint molecule in NK cells. CISH is rapidly induced in response to IL-15 and deletion of CISH renders NK cells hypersensitive to IL-15. The CISH knockout NK cells have enhanced proliferation, increased IFNγ production, and enhanced cytotoxic activity. Ablation of CIS releases a brake on NK cell activity.

The present NK cells may have disruption of CISH by any method known in the art, such as by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof. In particular aspects, CISH expression is disrupted by CRISPR-mediated disruption.

B. IL-15

Interleukin-15 (IL-15) is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits AICD.

In one embodiments, the present disclosure concerns co-modifying CAR and/or TCR immune cells with IL-15. In addition to IL-15, other cytokines are envisioned. These include, but are not limited to, cytokines, chemokines, and other molecules that contribute to the activation and proliferation of cells used for human application. NK or T cells expressing IL-15 are capable of continued supportive cytokine signaling, which is critical to their survival post-infusion.

In certain embodiments, K562 aAPC were developed, expressing the desired antigen (e.g., CD19) along with costimulatory molecules, such as CD28, IL-15, and CD3ζ, to select for immune cells (e.g., NK cells) in vitro that are capable of sustained CAR-mediated propagation. This powerful technology allows the manufacture of clinically relevant numbers (up to 10¹⁰) of CARP NK cells suitable for human application. As needed, additional stimulation cycles can be undertaken to generate larger numbers of genetically modified NK cells. Typically, at least 90% of the propagated NK cells express CAR and can be cryopreserved for infusion. Furthermore, this approach can be harnessed to generate NK cells to diverse tumor types by pairing the specificity of the introduced CAR with expression of the tumor-associated antigen (TAA) recognized by the CAR on the aAPC.

Following genetic modification the cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the chimeric receptor is expanded ex vivo. The clone selected for expansion demonstrates the capacity to specifically recognize and lyse CD19 expressing target cells. The recombinant immune cells may be expanded by stimulation with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

A. Genetically Engineered Antigen Receptors

The NK cells of the present disclosure can be genetically engineered to express antigen receptors such as engineered TCRs and/or CARs. For example, the NK cells are modified to express a TCR having antigenic specificity for a cancer antigen. Multiple CARs and/or TCRs, such as to different antigens, may be added to the NK cells.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

Electroporation of RNA coding for the full length TCR α and β (or γ and δ) chains can be used as alternative to overcome long-term problems with autoreactivity caused by pairing of retrovirally transduced and endogenous TCR chains. Even if such alternative pairing takes place in the transient transfection strategy, the possibly generated autoreactive T cells will lose this autoreactivity after some time, because the introduced TCR α and β chain are only transiently expressed. When the introduced TCR α and β chain expression is diminished, only normal autologous T cells are left. This is not the case when full length TCR chains are introduced by stable retroviral transduction, which will never lose the introduced TCR chains, causing a constantly present autoreactivity in the patient.

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

2. Chimeric Antigen Receptors

In some embodiments, the CAR comprises: a) an intracellular signaling domain, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the VH and VL chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, CD5, CD7, CD22, CD70, CD30, BCMA, CD25, NKG2D ligands, MICA/MICB, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CARP immune cells (Singh et al., 2008; Singh et al., 2011).

3. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

B. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

C. Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

D. Suicide Genes

The CAR of the immune cells of the present disclosure may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

E. Methods of Delivery

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

a. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

b. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

(i) Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

(ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

c. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

d. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

F. Modification of Gene Expression

In some embodiments, the immune cells of the present disclosure are modified to have altered expression of CISH. In additional embodiments, the cells may be further modified to have disrupted expression of glucocorticoid receptor and/or TGFβ receptor (e.g., TGFβ-RII). In one embodiment, the immune cells may be modified to express a dominant negative TGFβ receptor II (TGFβRIIDN) which can function as a cytokine sink to deplete endogenous TGFβ.

In some embodiments, the altered gene expression is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the alteration of the expression, activity, and/or function of the gene is carried out by disrupting the gene. In some aspects, the gene is modified so that its expression is reduced by at least at or about 20, 30, or 40%, generally at least at or about 50, 60, 70, 80, 90, or 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene alteration is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct.

1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed. (See, for example, Sigma-Aldrich catalog numbers CSTZFND, CSTZFN, CTil-1KT, and PZD0020).

2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et al., 2013). Custom-designed TALE arrays are commercially available through Cellectis Bioresearch (Paris, France), Transposagen Biopharmaceuticals (Lexington, Ky., USA), and Life Technologies (Grand Island, N.Y., USA). Specifically, TALENs that target CD38 are commercially available (See Gencopoeia, catalog numbers HTN222870-1, HTN222870-2, and HTN222870-3). Exemplary molecules are described, e.g., in U.S. Patent Publication Nos. US 2014/0120622, and 2013/0315884.

In some embodiments the TALEN s are introduced as trans genes encoded by one or more plasmid vectors. In some aspects, the plasmid vector can contain a selection marker which provides for identification and/or selection of cells which received said vector.

3. RGENs (CRISPR/Cas Systems)

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Exemplary gRNA sequences for NR3CS (glucocorticoid receptor) include Ex3 NR3C1 sG1 5-TGC TGT TGA GGA GCT GGA-3 (SEQ ID NO:1) and Ex3 NR3C1 sG2 5-AGC ACA CCA GGC AGA GTT-3 (SEQ ID NO:2). Exemplary gRNA sequences for TGF-beta receptor 2 include EX3 TGFBR2 sG1 5-CGG CTG AGG AGC GGA AGA-3 (SEQ ID NO:3) and EX3 TGFBR2 sG2 5-TGG-AGG-TGA-GCA-ATC-CCC-3 (SEQ ID NO:4). The T7 promoter, target sequence, and overlap sequence may have the sequence TTAATACGACTCACTATAGG (SEQ ID NO:5)+target sequence+gttttagagctagaaatagc (SEQ ID NO:6).

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### III. METHODS OF TREATMENT

In some embodiments, the present disclosure provides methods for immunotherapy comprising administering an effective amount of the NK cells of the present disclosure. In one embodiment, a medical disease or disorder is treated by transfer of an NK cell population that elicits an immune response. In certain embodiments of the present disclosure, cancer or infection is treated by transfer of an NK cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

B. Pharmaceutical Compositions

Also provided herein are pharmaceutical compositions and formulations comprising immune cells (e.g., T cells or NK cells) and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

C. Combination Therapies

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. ARTICLES OF MANUFACTURE OR KITS

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—CAR NK Cells with Knockout of CISH

NK cells were isolated by negative selection from 3 different cord blood (CB) units and expanded. The NK cells were then transduced with the CAR construct and CISH KO was performed for each CB at different time points. Four different groups of cells were obtained for each CB unit including NT, NT CISH KO, CAR19/IL-15, and CAR19/IL-KO.

Specifically, on Day 1, NK were isolated using negative selection, cocultured with irradiated APCs (K562 expressing 41BB and IL-21 on their surface) at a 1:2 ratio (NK:APC) in SCGM with IL-2 200 units/ml. The media was changed every 2 days with fresh SCGM and IL-2 200 units/ml.

On Day 5, the NK cells were reselected to obtain pure NK cells and perform CAR transduction was performed. At this time, both non-transduced cells (NT) and CAR NK cells were present. CAR transduction efficiency was measured 3 days post-transduction.

On Day 7, irradiated APCs were added to re-expand the NK cells (both NT and CAR NK). The NK cells were cultured in SCGM with IL-2 200 U/ml. The media was changed every 2 days with fresh SCGM and IL-2 200 U/ml.

On Day 14, CRISPR Cas9 was used for CISH KO in CAR NK cells using Neon electroporation. Two sgRNAs were designed targeting exon 4 of the CISH gene.

About 15-30 mins before electroporation, the Cas9 protein and sgRNA were incubated at room temperature in a 1:1 reaction with 10 ug Cas9 and 10 ug sgRNA (5000 ng of sgRNA #1 and 5000 ng of sgRNA #2). The incubation product (i.e., sgRNA and Cas9 bound together) was electroporated into 1-2 million cells with a 100 uL electroporation tip. The electroporation conditions were 1600 V, 10 ms, 3 pulses, using the T buffer. The different cell preparations (electroporated CAR NK cells, control CAR NK cells and NT NK cells) were co-cultured with irradiated APCs at 1:2 ratio (NK:APC) in SCGM with IL-2 20 U/mL. The media was changed every 2 days with fresh SCGM and IL-2 200 U/ml. On Day 15, PCR was performed to check KO efficiency.

Finally, functional studies were performed on day 21. The cells from the 4 groups were co-cultured in 96 well plate with BFA and Raji at 2:1 ratio for 6 hours Staining was then performed to compare cytokine production (IFNγ, TNFα and CD107α) which were evaluated by flow cytometry. Chromium release assay was performed on day 21 to compare the cytotoxicity of the 4 different groups of cells for each CB. Statistical analysis was done using two-way Anova with multiple comparisons using the Bonferroni test.

Gene editing of NK cells is more challenging than that of T cells. Unlike T cells, NK cells need feeder cells to grow well in vitro which makes the process of gene editing more complex. NK cells are also more fragile and historically have been known to have poor viability after genetic manipulation. The present viability after CAR transduction and CISH KO was more tan 95%.

### Example 2—In Vivo Characterization of NK Cells

To characterize the CAR NK cells with CISH KO in vivo, 10 week old NSG null, female mice were studied. The mice were injected with 20×10³ Raji-1-1-Luc cells. For the effector cells, Cell #1-Cell #3, 3×10⁶ or 10⁷ cells/animal were injected depending on the groups below.

Group Categories: 5 mice/group, 7 groups and 35 mice total,

Group #1: Raji-FFLuc Only

Group #2: Raji-FFLuc+Cells #1 3×10⁶ cells/animal

Group #3: Raji-FFLuc+Cells #1 10⁷ cells/animal

Group #4: Raji-FFLuc+Cells #2 3×10⁶ cells/animal

Group #5: Raji-FFLuc+Cells #2 10⁷ cells/animal

Cell #1: NKCAR19 cas9+electroporation

Cell #3: NKCAR19 CISH KO

Mice were administered Raji cells alone or in combination with CAR NK cells. The CAR NK cells were wild-type, cas9 only, or had knockout of CISH. It was found that mice that received the CISH knockout CAR NK cells had increased survival, showed no evidence of Raji lymphoma, and showed no CRS. The CISH knockout NK cells also persisted longer in vivo and could be detected up to 7 weeks post-infusion.

### Example 3—CIS Checkpoint Deletion Modulates the Fitness of Cord Blood Derived CAR-Transduced Natural Killer Cells

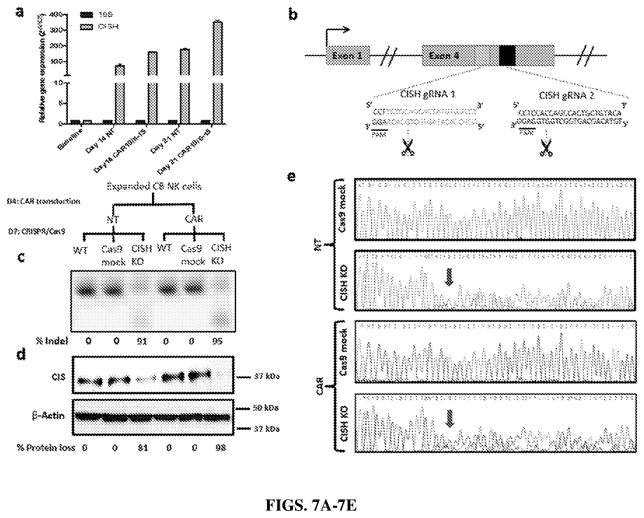

Enhanced in vitro functionality in CIS deficient iC9/CAR19/IL-15 NK cells: The expression levels of CIS were evaluated to determine if iC9/CAR19/IL-15 CB-NK cells are subject to the same counter regulatory circuits that physiologically down-regulate cytokine signaling in unmodified NK cells. CB-NK cells were cultured with a K562 feeder cell line engineered to express membrane-bound IL21, 4-1BB ligand and CD48 (uAPC) in the presence of IL-2 for 21 days. NK cells were either transduced on day +4 with a retroviral vector expressing iC9/CAR19/IL-15 as described in materials and methods or were not transduced (NT, control). CIS expression increased significantly during in vitro expansion of NT control and iC9/CAR19/IL-15 CB-NK cells over time (FIG. 7A). Notably, CIS expression levels were more prominent in iC9/CAR19/IL-15 compared to NT CB-NK cells likely due to the additional effect of IL-15 on CIS induction (FIG. 7A).

The functional consequences of CISH deletion were next examined in iC9/CAR19/IL-15 CB-NK cells. Since CIS is a potent immune checkpoint in NK cells, it was hypothesized that knocking out the CISH gene could enhance their effector function in a way similar to PD-1 blockade in T cells. To test this idea, a protocol was first developed for combined retroviral transduction with the iC9/CAR19/IL15 construct and Cas9 ribonucleoprotein (Cas9 RNP)-mediated gene editing to silence CISH. On day 7 CAR transduced NK cells were nucleofected with Cas9 alone (Cas9 mock) or Cas9 pre-loaded with crRNA:tracrRNA duplex targeting CISH exon 4 (CISH KO) (FIGS. 7A-B). The iC9/CAR19/IL-15 transduction efficiency and cell viability on day 7 were greater than 90% and remained stable over time (FIG. 13). CISH KO efficiency was greater than 80% in both the NT and CAR-expressing NK cells as assessed by polymerase chain reaction (PCR) (FIG. 7A) and western blot analysis (FIG. 7B). These findings were also confirmed by Sanger sequencing (FIG. 7C).

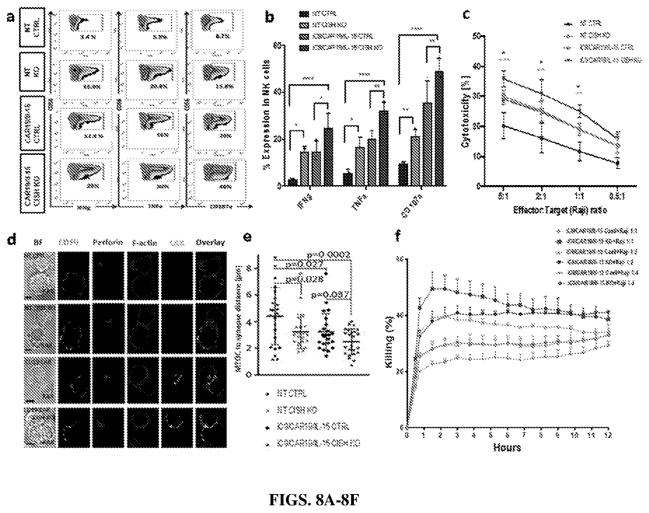

To determine the effect of CISH KO on the antitumor activity of NK cells, the response of NT and iC9/CAR19/IL-15 CB-NK cells was tested seven days after electroporation with Cas9 coupled with a non-target gRNA (CTRL) or with Cas9 coupled with gRNA targeting the CISH gene (CISH KO) against Raji lymphoma cell targets. CISH KO CAR.NK cells produced significantly higher amounts of IFN-γ and TNF-α, displayed enhanced degranulation (CD107a) and exerted greater cytotoxicity against Raji compared to their respective NT or iC9/CAR19/IL-15 controls (FIGS. 8A-8C). Notably, among all groups CISH KO iC9/CAR19/IL-15 CB-NK cells displayed the highest cytokine production and cytotoxicity against Raji targets, supporting the idea that combining CAR transduction with CISH silencing results in enhanced NK cell function (FIGS. 8A-8C).

The effect of CISH KO was then examined on the immunologic synapse (IS) formation between NT or iC9/CAR19/IL-15 NK cells and Raji cells. The polarization of the microtubule organizing center (MTOC) was augmented by CISH KO as reflected by a shortened MTOC to IS distance compared to controls (FIG. 8E). Taken together, these findings suggest that CISH KO strengthens the immunologic synapse between NK cells (NT and iC9/CAR19/IL-15) and tumor cells, which correlates with increased effector function and cytotoxicity.

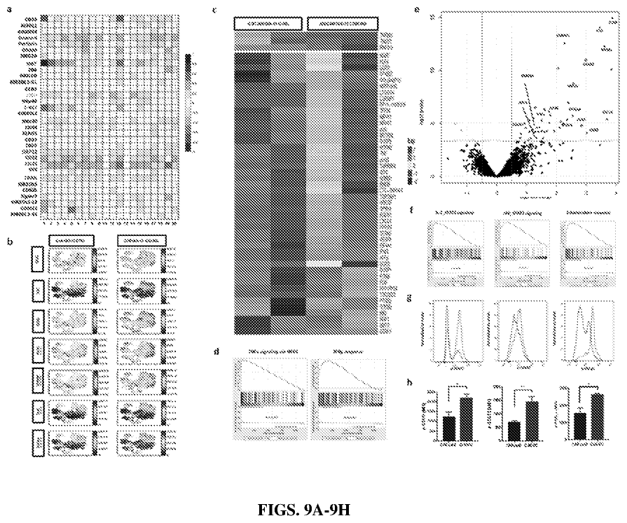

Phenotypic and molecular signature of CISH KO NT and iC9/CAR19/IL-15 transduced NK cells: To understand the mechanism by which CISH KO in NK cells increases their function against tumor cells, cytometry by time-of-flight (CyToF) was used and a panel of 32 antibodies against inhibitory and activating receptors, as well as differentiation, homing and activation markers, to gain insights into their phenotypic composition. CISH KO in iC9/CAR19/IL-15 NK cells induced a functional phenotype with significantly higher expression of markers of cytotoxicity including granzyme-b, perforin, TRAIL, CD3z, transcription factors and adaptor molecules such as Eomes, T-bet, DAP12, and activating coreceptors/proliferation markers such as DNAM, CD25 and Ki67 compared to their control counterpart (FIG. 9A). Similarly, deletion of CISH in NT NK cells resulted in upregulation of several activation markers compared to control NT NK cells (FIG. 14A). viSNE, a t-distributed stochastic neighbor embedding (tSNE) algorithm was used to further analyze these activated NK cells markers following CISH KO and observed considerable segregation between control and CISH KO iC9/CAR19/IL-15 NK cells, with a predominantly activated phenotype (increased expression of CD25, Ki67, CD3z, perforin and granzyme-b) dominating the CISH KO iC9/CAR19/IL-15 NK cells (FIG. 9B).

RNA sequencing was next performed to gain a deeper understanding of the NK cell transcriptomic profile following CISH KO. Notably, the results revealed distinct gene expression profile between CISH KO and control iC9/CAR19/IL-15 NK cells (FIG. 9C). CISH KO in NT NK cells led to upregulation of a limited number of genes related to interferon stimulated genes (ISGs) and STAT-1 including OSA-1, OSA-2 (FIGS. 14A-14C). In contrast, CISH KO in iC9/CAR19/IL-15 NK cells led to upregulation of multiple genes related to JAK/STAT activation, and the MAPK/ERK pathway (FIG. 9).

To gain greater insight into the mechanistic basis for the increased function and cytotoxicity induced by CISH KO in iC9/CAR19/Il-15 NK cells gene set enrichment analysis (GSEA) was used to identify sets of genes or biological pathways that could contribute to the phenotype. The analysis revealed enrichment in TNF-α, IFN-γ, IL-2/STAT5 and IL-6/STAT3 signaling, as well as genes related to inflammatory response (FIG. 9F). The findings were confirmed at the protein level by showing enhanced phosphorylation of STAT5, STAT3 and Phospholipase C gamma 1 (PLCg1) in CISH KO iC9/CAR19/Il-15 CB-NK cells (FIG. 9G, H). Taken together, these data indicate that IL-15 drives activation of CISH, and that CISH KO in iC9/CAR19/IL-15 NK cells induces a molecular signature compatible with an activated phenotype.

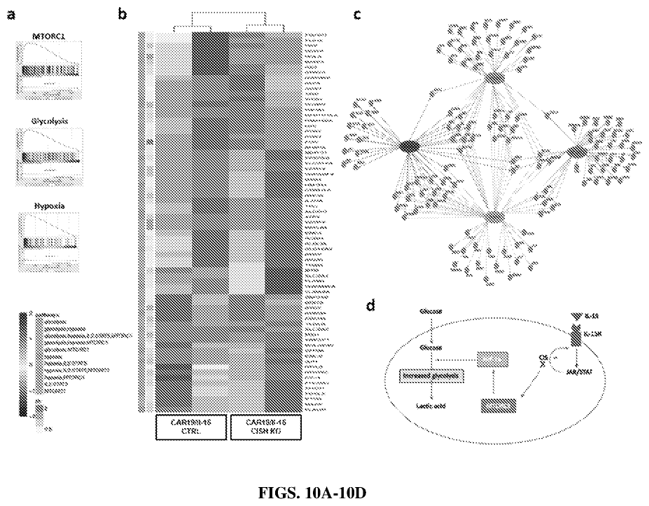

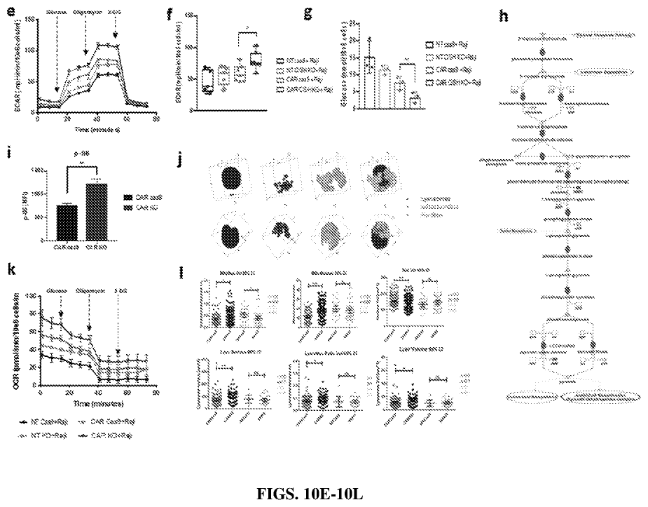

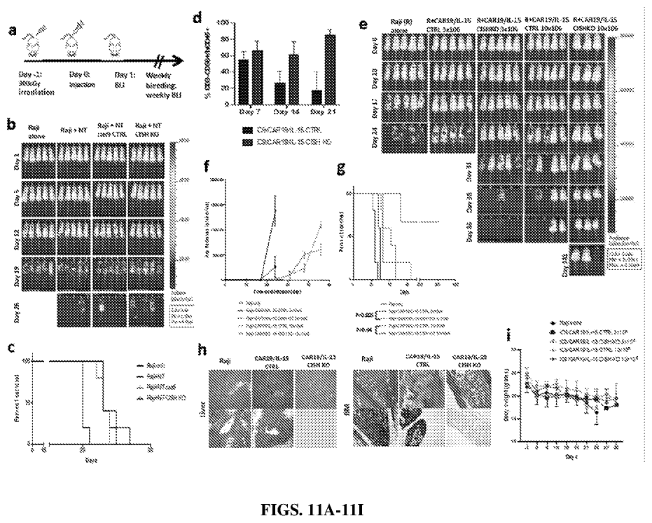

CISH KO reprograms the metabolism of iC9/CAR19/IL-15 NK cells: As shown in FIG. 10A, RNA seq data and GSEA results also showed the novel observation that CISH KO results in enrichment in mTORC1, hypoxia and glycolysis genes. The activity of mTORC1 is essential for glycolytic reprogramming of activated NK cells and has been asserted as a prerequisite for effector NK cell functions. Thus, it was sought to determine whether deletion of CISH modulates the metabolic activity of iC9/CAR19/IL-15 NK cells. It was hypothesized that CISH KO enhances the cytotoxic activity of iC9/CAR19/IL-15 NK cells by modulating their metabolism and increasing oxygen and glucose consumption. To test this hypothesis, a series of Seahorse assays were performed to measure the energy pathways in NK cells and showed that in response to Raji lymphoma, CISH KO iC9/CAR19/IL-15 NK cells had a greater glycolytic reserve as measured by higher extracellular acidification rate (ECAR) when compared to Cas9 control iC9/CAR19/IL-15 NK cells. These functional data were further supported by ingenuity pathway analysis (IPA) showing upregulation of glycolysis enzymes. In addition, CISH KO iC9/CAR19/IL-15 NK cells displayed higher glucose consumption compared to their Cas9 controls as demonstrated by glucose colorimetric test performed on the supernatant of CAR-NK cells co-cultured with Raj i tumor cells for 3 hrs. This suggests that CISH KO increased the metabolic activity of CAR-NK cells by enhancing their ability to consume glucose and utilize it for glycolysis. Furthermore, CISH KO iC9/CAR19/IL-15 NK cells demonstrated increased oxygen consumption rate (OCR) by Seahorse assay compared to control iC9/CAR19/IL-15 NK cells. This indicates that CISH KO also enhances the metabolism of CAR-NK cells by revving up their mitochondrial activity. Indeed, in a series of specialized confocal microscopy studies, it was observed that the number of mitochondria and the mitochondrial/nuclear volume ratio are significantly higher in iC9/CAR19/IL-15 CISH KO cells compared to Cas9 controls (FIG. 10).

Based on these data, it was hypothesized that by increasing JAK/STAT activation, CISH KO leads to enhanced MTORC1 activity and upregulation of HiF1a activity, culminating in increased glycolytic capacity and NK cell fitness. Indeed, it was found that following co-culture with Raji cells for 2 hrs, phosphorylation of the ribosomal protein S6 (S6), a downstream target of mTOR1 pathway activation, was significantly higher in CISH KO iC9/CAR19/Il-15 compared to Cas9 controls.

Combination of CIS checkpoint disruption and iC9/CAR19/IL-15 engineering improves tumor control and survival in a Raji lymphoma mouse model: Using an aggressive Raji lymphoma NSG mouse model (FIG. 11A), it was investigated whether adoptive transfer of CISH KO CB-NK cells could augment their in vivo tumor control. First, mice received one i.v infusion (10×10⁶/mouse) of control NT CB-NK cells that were either electroporated with Cas9 (Cas9 CTRL) or had CISH KO (5 mice per group). Tumor growth was monitored using changes in tumor bioluminescence imaging (BLI) over time. Tumor burden increased over time with no significant difference in survival between the groups (FIGS. 11B-C), suggesting that in the absence of CAR transduction, CISH KO does not enhance the activity of NK cells against Raji tumor. The in vivo effect of CISH KO was next investigated on the antitumor activity of iC9/CAR19/IL-15 NK cells. Since CISH KO iC9/CAR19/IL-15 cells are more potent at killing target tumor cells even at low E:T ratios, it was hypothesized that they will also be more effective at controlling the tumor at lower infusion doses. Indeed, infusion of as few as 3×10⁶ cells, CISH KO iC9/CAR19/IL-15 NK cells significantly improved survival and control of Raji lymphoma compared to CTRL iC9/CAR19/IL-15 CB-NK cells, but eventually mice succumb to tumor after 46 days (FIGS. 11E-G). When a higher dose of 10×10⁶ CAR-NK cells were infused, the group that received CISH KO cells did not show any evidence of tumor by BLI (FIG. 11) or on pathology and showed an impressively prolonged survival for up to 341 days compared to CTRL iC9/CAR19/IL-15 CB-NK cells (FIGS. 11E-H, 16). This was associated with improved NK cell persistence in mice that received CISH KO iC9/CAR19/IL-15 compared to animals that received CTRL iC9/CAR19/IL-15 CB-NK cells (FIG. 11D). Of note, CISH KO was not associated with signs of increased toxicity in mice, such as weight loss (FIG. 11I). Together, these data indicate that silencing CISH improved the ability of iC9/CAR19/IL-15 CB-NK cells to exert in vivo control of the tumor in vivo without increasing toxicity.

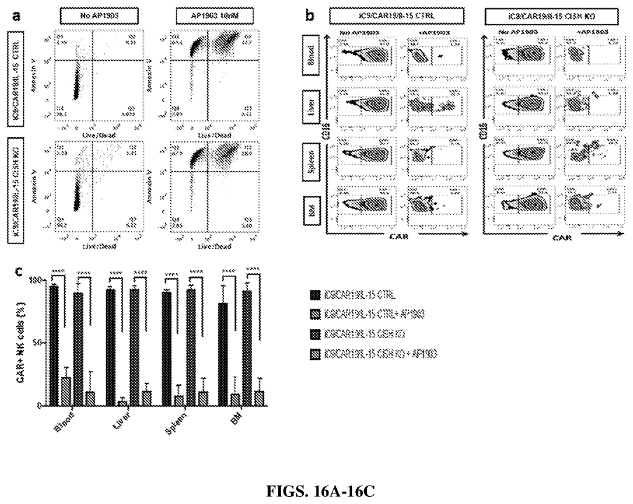

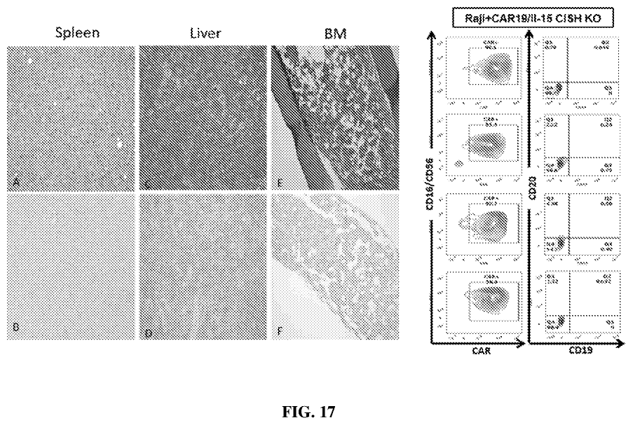

Clinical translation of off-the-shelf CISH KO iC9/CAR19/IL-15 NK cells for relapsed/refractory B cell malignancy: Even though increased in vivo toxicity was not encountered in mice treated with CISH KO CAR.NK cells, enhancing IL-15 signaling may potentially result in increased release of inflammatory cytokines. Thus, inducible caspase 9 (iC9) was used as a suicide gene in the construct to confirm that CISH KO CAR-transduced NK cells could be induced to undergo apoptosis in presence of a small-molecule dimerizer AP1903. The addition of as little as 10 nM of AP1903 to cultures of iC9/CAR.19/IL-15 NK cells induced apoptosis of transduced NK cells within 4 h, and CISH KO did not affect the action of the dimerizer (FIG. 17A). The suicide gene was also effective at eliminating the CAR cells in vivo in both CISH KO and control iC9/CAR.19/IL-15 CB-NK cells (FIG. 16B). Mice engrafted with Raji tumor received either control or CISH KO iC9/CAR.19/IL-15 CB-NK cells followed by treatment with the dimerizer on days 7 and day 9 post NK infusion (n=5 mice per group). The animals were then sacrificed on day 12. Administration of the small-molecule dimerizer resulted in a striking reduction in iC9/CAR.19/IL-15 CB-NK cells (both control and CISH KO) in the blood and tissues (liver, spleen and bone marrow) of the treated mice (FIGS. 17B-C).

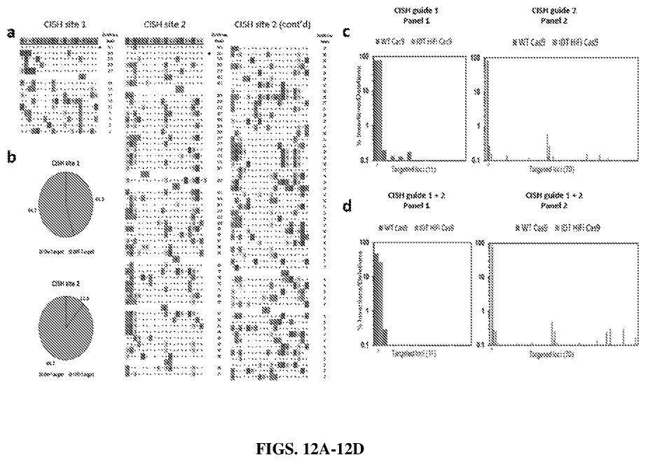

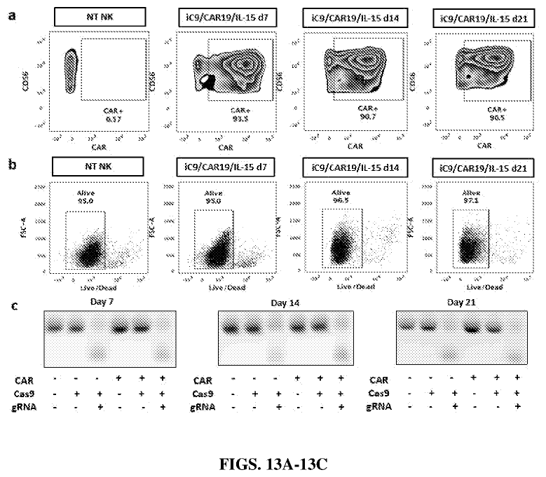

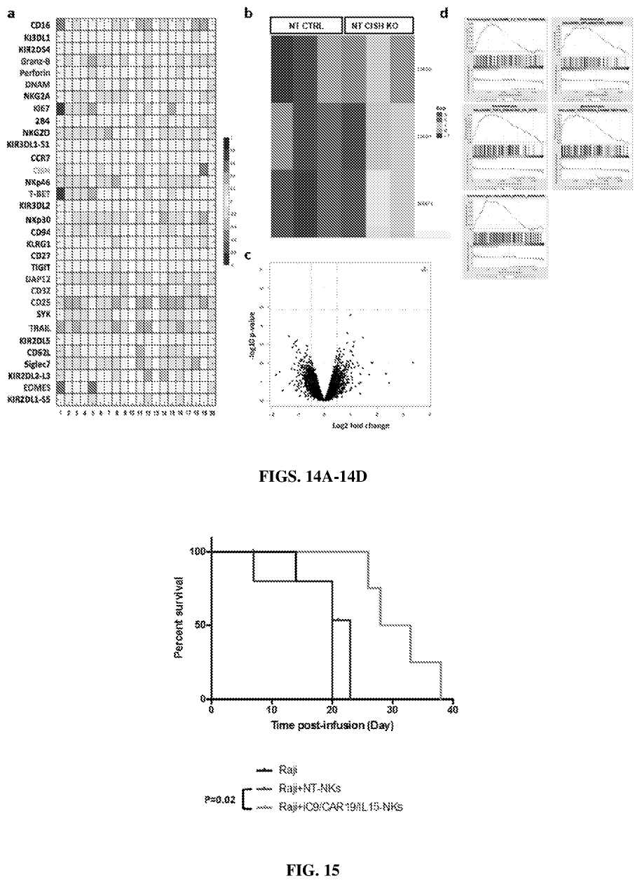

Identification of off-target CRISPR RNA-guided nuclease (RGN) insertions/deletions (INDELs) is crucial before this approach can be taken to the clinic. Thus, GuideSeq and Rhampseq technologies were used to evaluate the off-target effects of the specific CISH guide RNAs used in the experiments. GuideSeq experiments using HEK293 cells that constitutively express Cas9 nuclease identified multiple potential off-target sites for both crRNAs targeting the CISH locus, and even higher frequencies of off-target events were noted with crRNA2 (FIGS. 12A-B).

In summary, this is the first report of a genetic engineering strategy combining CAR transduction and checkpoint blockade in cord blood derived NK cells. This NK cellular therapy product is off the shelf, safe and potent at eliminating CD19+ tumor cells even at low doses.

### Example 4—Material and Methods

Cell lines and culture media: Raji (Burkitt lymphoma cell line) was purchased from American Type Culture Collection (Manassa, Va., USA). K562 based feeder cells were retrovirally transduced to co-express 4-1BBL, CD48 and membrane bound IL-21 9uAPCs). Firefly luciferase transduced Raji (Raji-FFLUC) cells used for the in vivo experiments were kindly provided by Dr. Gianpietro Dotti (University of North Carolina).

All cell lines were cultured in Roswell Park Memorial Institute (RPMI) medium supplemented with 5% Fetal Bovine Serum (FBS), 1% Penicillin-Streptomycin and 1% L-Glutamine NK cells were cultured in Stem Cell Growth Medium (SCGM) supplemented with 5% Fetal Bovine Serum (FBS), 1% Penicillin-Streptomycin and 1% L-Glutamine

Cord blood NK cell expansion: CB units for research were provided by the MD Anderson Cancer Center CB Bank. CB's were isolated by a density-gradient technique (Ficoll-Histopaque; Sigma, St Louis, Mo., USA). CD56+NK cells, purified with an NK isolation kit (Miltenyi Biotec, Inc., San Diego, Calif., USA), were stimulated with irradiated (100 Gy) uAPC (2:1 feeder cell:NK ratio) and recombinant human IL-2 (Proleukin, 200 U/ml; Chiron, Emeryville, Calif., USA) in complete stem cell growth medium (CellGenix GmbH, Freiburg, Germany) on day 0. Activated NK cells were transduced with retroviral supernatants on day +4 in human fibronectin-coated plates (Clontech Laboratories, Inc., Mountain View, Calif., USA). On day +7 and day +14, NK cells were stimulated again with irradiated uAPC and IL-2. On day +21, CAR-transduced NK cells were collected for use.

Retrovirus transfection and transduction: The retroviral vectors encoding iC9.CAR19.CD28-zeta-2A-IL-15 has been previously described (Vera et al., 2006). Transient retroviral supernatants were produced as previously described (Vera et al., 2006). Activated NK cells were transduced with retroviral supernatants on day +4 in human fibronectin-coated plates (Clontech Laboratories, Inc., Mountain View, Calif., USA). Three days later (day +7), CAR transduction efficiency was measured by flow cytometry and NK cells were stimulated again with irradiated uAPC and IL-2.

CRISPR/Cas9 gene editing of CISH: CISH KO was performed on day +7 using ribonucleoprotein (RNP) complex, in both NT and CAR transduced NK cells. Protospacer sequences for the CISH gene were identified using the CRISPRscan (Moreno-Mateos et al., 2015). DNA templates for gRNAs were made using the protocol described by Li et al. Two gRNAs were used targeting exon 4 of CISH gene: gRNA1:AGGCCACATAGTGCTGCACA (SEQ ID NO:1), gRNA2: TGTACAGCAGTGGCTGGTGG (SEQ ID NO:2). Cas9 protein (PNA bio) and gRNA were incubated at room temperature for 15 min in a 1:1 reaction with bug Cas9 and Mug gRNA (5000 ng of sgRNA #1 and 5000 ng of sgRNA #2). The incubation product (gRNA and Cas9 bound together) was then used to electroporate 1-2 million NT or CAR transduced NK cells using Neon transfection system (Thermo Fisher Scientific). Optimized electroporation conditions were 1600V, 10 ms, 3 pulses, using T buffer. The different cell preparations were then co-cultured with irradiated uAPC at 1:2 ratio (NK:uAPC) in SCGM media with IL-2 200 U/ml.

Real-time quantitative PCR (RT Q-PCR): Total RNA was isolated using the RNeasy Plus Mini kit (Qiagen Inc., Hilden, Germany) and cDNA synthesis performed with ReadyScript cDNA Synthesis Mix (Sigma-Aldrich Corp., St. Louis, Mo., USA) according to the manufacturer's instructions. PCR reactions were performed in 20 μL; 10 μL of TaqMan 2× Advanced Fast PCR MasterMix (Applied Biosystems Inc., Foster City, Calif., USA), 1 μL of CISH TaqMan Gene Expression Assay (Applied Biosystems), 2 μL of cDNA and 7 μL of nuclease-free water. Primer sequences and PCR conditions have been described (Kolesnik et al., 2013). Real-time Q-PCR was performed on an ABI 7500 Fast Real Time PCR System (Applied Biosystems). mRNA levels were quantified against standard curves generated using sequential dilutions of an oligonucleotide corresponding to each amplified PCR fragment and using 7500 Fast v2.3 Software (Applied Biosystems). Relative expression was determined by normalizing the amount of each gene of interest to the housekeeping gene 18S.

Western blot: NK cells were pre-treated with 10 μM MG132 for 4 h to block proteasomal degradation. NK cells were then lysed in lysis buffer (IP Lysis Buffer, Pierce Biotechnology Inc., Rockford, Ill.) supplemented with protease inhibitors (Complete Mini, EDTA-free Cocktail tablets, Roche Holding, Basel, Switzerland) and incubated for 30 min on ice. Protein concentrations were determined by the BCA assay (Pierce Biotechnology Inc., Rockford, Ill.). The following primary antibodies were used: CIS antibody (Clone D4D9) and B-actin antibody (Clone 8H10D10), both antibodies were obtained from Cell Signaling Technology.

Measuring allele modification frequencies using PCR and Sanger sequencing: DNA was extracted and purified (QIAamp DNA Blood Mini Kit, Qiagen Inc., Hilden, Germany from CAR-transduced and ex vivo-expanded NT NK cells (control and CISH KO conditions). The PCR primers used for the amplification of the target locus were as follows:

Exon 4 Forward Primer: CGTCTGGACTCCAACTGCTT (SEQ ID NO:7)

Exon 4 Reverse Primer: GTACAAAGGGCTGCACCAGT (SEQ ID NO:8)

Purified PCR products were sent for Sanger Sequencing at MD Anderson's core facility using both PCR primers, and each sequence chromatogram was analyzed.

Functional assay: On day 14 or day 21 of culture, control ex vivo-expanded NT (Control or CISH KO) and CAR19/Il-15 NK cells (control or CISH KO) at 100×10e3 cells/well were co-cultured in the presence of Brefeldin A for 6 h in round bottom 96-well plates with Raji cells or K562 targets (positive control) at an effector:target cell ratio (E:T) of 2:1. To measure degranulation, CD107a antibody (Brilliant Violet 785™ anti-human CD107a (LAMP-1) Antibody, Biolegend, San Diego, Calif., USA) was added to the wells at the beginning of co-culture. Degranulation measured by CD107a and intracellular cytokine production (TNFα (TNF alpha Monoclonal Antibody (MAb11), Alexa Fluor 700, eBioscience Inc., San Diego, Calif., USA) and IFN-γ (BD Horizon™ V450 Mouse Anti-Human IFN-γ, BD Biosciences, San Jose, Calif., USA) were assessed by flow cytometry as previously described (Rouce et al., 2016)

Chromium Release assay: To assess cytotoxicity, ex-vivo expanded NT NK cells (control and CISH KO) and CAR-transduced (control and CISH KO) were co-cultured with ⁵¹Cr-labeled Raji targets at multiple E:T ratios; cytotoxicity was measured by ⁵¹Cr release as previously described (Rouce et al., 2016).

Confocal microscopy for studying the immunologic synapse: 0.5×10⁶ effector cells, as described above, were conjugated with 0.25×10⁶ Raji cells in 250 ml of SCGM with 10% heat inactivated FBS containing media, for 40 minutes at 37° C. and stained as demonstrated elsewhere (Banarjee et al., 2010). Briefly, after incubation, cells were adhered onto a Poly-A-Lysine coated slide (Electron Microscopy Sciences) and stained for proteins of interest. Alexa Fluor 647-conjugated affinity-purified F(ab′)2 fragment goat anti-human IgG (H+L) antibody was used to detect CAR. Anti-CD19 Alexa Flour (AF) 488 (clone HIB19, BD BioSciences), Phalloidin AF 568 (Invitrogen) for detection of F-actin, and anti-Perforin 488 (clone δ G9; BioLegend) were used. Conjugates were mounted in anti-fade containing media (Prolong gold, Invitrogen) and were imaged by sequential scanning with a Yokogawa spinning disk confocal microscope equipped with a Zyla 4.2sCMOS Camera, and under 63× objective. Images were exported to Imaris (Bitplane) for quantitative measurements. The distance from perforin centroid to synapse is measured as previously described (.

Mass Cytometry

Antibody conjugation: A panel comprising of 38 metal-tagged antibodies was used for the in-depth characterization of NK cells. The list of antibodies with the corresponding metal tag isotopes. All unlabeled antibodies were purchased in carrier-free form and conjugated in-house with the corresponding metal tag using Maxpar X8 polymer per manufacturer's instructions (Fludigm). All metal isotopes were acquired from Fludigm except for indium (III) chloride (Sigma-Aldrich, St. Louis, Mo.). Antibody concentration was determined by measuring the amount of A280 protein using Nanodrop 2000 (Thermo Fisher Scientific, Waltham, Mass.). Conjugated antibodies were then diluted in PBS-based antibody stabilization solution or LowCross-Buffer (Candor Bioscience GmbH, Wangen, Germany) supplemented with 0.05% sodium azide (Sigma-Aldrich, St. Louis, Mo.) to a final concentration of 0.5 mg/ml. Serial titration experiments were performed to determine the concentration with the optimal signal to noise ratio for each antibody.

Sample preparation and acquisition: NT NK cells (Control or CISH KO) and CAR transduced NK cells (Control or CISH KO) were harvested, washed twice with cell staining buffer (0.5% bovine serum albumin/PBS) and incubated with 5 μl of human Fc receptor blocking solution (Trustain FcX, Biolegend, San Diego, Calif.) for 10 minutes at room temperature. Cells were then stained with a freshly prepared antibody mix against cell surface markers for 30 minutes at room temperature on a shaker (100 rpm). For the last 3 minutes of incubation, cells were incubated with 2.5 μM cisplatin (Sigma Aldrich, St Louis, Mo.), washed twice with cell staining buffer and fixed/permeabilized using BD Cytofix/Cytoperm solution for 30 minutes in dark at 4° C. Cells were washed twice with perm/wash buffer, stained with antibodies directed against intracellular markers and after an additional wash step, stored overnight in 500 μl of 1.6% paraformaldehyde (EMD Biosciences)/PBS with 125 nM iridium nucleic acid intercalator (Fluidigm). The next day, samples were washed twice with cell staining buffer, resuspended in 1 ml of MilliQ dH2O, filtered through a 35 μm nylon mesh (cell strainer cap tubes, BD, San Jose, Calif.) and counted. Before analysis, samples were suspended in MilliQ dH2O supplemented with EQ™ four element calibration beads at a concentration of 0.5×105/ml. Samples were acquired at 300 events/second on a Helios instrument (Fluidigm) using the Helios 6.5.358 acquisition software (Fluidigm).

Data analysis: Mass cytometry data were normalized based on EQ™ four element signal shift over time using Fluidigm normalization software 2. Initial data quality control was performed using Flowjo version 10.2. Calibration beads were gated out and singlets were chosen based on iridium 193 staining and event length. Dead cells were excluded by the Pt195 channel and further gating was performed to select CD45+ cells and then the NK cell population of interest (CD3−CD56+). A total of 320,000 cells were proportionally sampled from all samples to perform automated clustering. Data were analyzed using automated dimension reduction including (viSNE) in combination with FlowSOM for clustering for the deep phenotyping of immune cells as published before (Van Gassen et al., 2015).

RNA sequencing: RNA was extracted and purified (RNeasy Plus Mini Kit, Qiagen) from CAR-transduced and ex vivo-expanded NT NK cells (control and CISH KO conditions) and sent for RNA seq to Novogene, where quality control, library construction and sequencing were performed. Analysis of RNAseq data was performed by MD Anderson Bioinformatics department. Sequencing reads were aligned to human reference genome (hg38) using TOPHAT2 v2.0.13 (Kim et al., 2013). The gene expression levels were measured by counting the mapped reads using HTSEQ based on hg38 GENCODE v25 gene model. The differentially expressed genes were identified using EdgeR package, with FDR (false discovery rate) cutoff <0.01 and fold change >2 (Robinson et al., 2010).

Mitochondria and Lysosome Imaging

Labeling NK cells: NK cells were incubated at 37° C. for 40 minutes in 1:1 (v/v) solution of live cell staining buffer (abcam) and RPMI (Corning) containing final concentrations of 500 nM MitoTracker™ Deep Red FM (Invitrogen™), 250 nM LysoRed (abcam) and 1 μM Hoechst 33342 (Sigma) for labeling mitochondria, lysosome and nucleus respectively. Cells then were washed with Hanks' balanced salt solution (Cellgro)+10% HEPES (Corning) first and complete culture medium RPMI+10% FBS (R10) for the second wash.

Confocal microscopy: 50,000 of NK cells were loaded in individual wells of 96 glass-bottom plate. A Nikon A1/TiE inverted microscope fitted with a 100×, 1.45 NA objective was used for imaging. 3D images (z-stacks, 0.3 μm steps, ˜40 slices) were taken from different field of views using DAPI (404.0 nm), TXRed (561.8 nm) and Cy5 (641.0 nm) channels.

Analyzing confocal images: Z-stacks of 16-bit images were extracted for each channel and processed in ImageJ (National Institutes of Health (NIH), USA) using a series of plugins. First, images were segmented for each channel prior to applying a threshold. Next, the 3D Objects Counter plugin was applied to the image to determine mitochondrial and lysosome regions of interest (ROIs). These ROIs were overlaid onto the original image and measurements were collected afterward. Similarly, the 3D Objects Counter plugin was also used on nucleus but using the original image only. Lastly, tracking of single cell movement was done using the TrackMate 1 plugin in order to filter out unstable cells upon their movement. All measurements were consolidated in R, where mitochondria, lysosome and nucleus were matched to their corresponding cell.

Xenogeneic lymphoma models: To assess the antitumor effect of CAR-transduced CB-NK cells in vivo, we used a NOD/SCID IL-2Rγnull (NSG) xenograft model, with the aggressive NK-resistant Raji cell line. Mouse experiments were performed in accordance with NIH recommendations under protocols approved by the Institutional Animal Care and Use Committee. NSG mice (10-12 weeks old; Jackson Laboratories, Bar Harbor, Me., USA) were irradiated with 300 cGy at day-1 and inoculated intravenously with firefly luciferase-labeled Raji cells (2×10e4) on day 0. Where indicated, fresh expanded NT (control or CISH KO) or CAR-transduced CB-NK (Control or CISH KO) cells were injected through the tail vein on day 0. Mice were subjected to weekly bioluminescence imaging (Xenogen-IVIS 200 Imaging system; Caliper, Waltham, Mass., USA). Signal quantitation in photons/second was performed by determining the photon flux rate within standardized regions of interest using Living Image software (Caliper). Trafficking, persistence and expansion of NK cells were measured by flow cytometry.

Activation of suicide gene in vitro and validation in vivo: The small-molecule dimerizer AP1903 (10 nM), was added to CB-NK cell cultures for 4 h. The elimination of transduced cells was evaluated by Annexin-V/Live dead staining. The efficacy of the suicide gene was also tested in vivo by treating tumor-bearing mice that had received iC9/CAR.19/IL-15+ NK cells (control or CISH KO) with two doses of AP1903 (50 μg each) intraperitoneally, 2 days apart on days 7 and 9. Two other groups of mice (control and CISH KO) served as control without AP1903 treatment. All mice were sacrificed on day 12, and blood and organs (Liver, spleen and bone marrow) were collected, processed and flow cytometry was performed to determine CAR-NK fraction and viability.

Off-target Identification: The GUIDE-seq method was employed for unbiased discovery of off-target editing events (Tsai et al., 2015). In this study, HEK293 cells that constitutively express the S pyogenes Cas9 nuclease (“HEK293-Cas9” cells) were the source of Cas9. Alt-R® gRNA complexes were formed by combining Alt-R tracrRNA and Alt-R crRNA XT at a 1:1 molar ratio. gRNA complexes were delivered by nucleofection using the Amaxa™ Nucleofector™ 96-well Shuttle™ System (Lonza, Basel, Switzerland). For each nucleofection, 3.5×10⁵ HEK293-Cas9 cells were washed with 1×PBS, resuspended in 20 μL solution SF (Lonza) and combined with 10 μM gRNA along with 0.5 μM GUIDE-seq dsDNA donor fragment. This mixture was transferred into one well of a Nucleocuvette™ Plate (Lonza) and electroporated using protocol 96-DS-150. DNA was extracted 72 hrs after electroporation using the GeneJET Genomic DNA purification kit (Thermo Fisher Scientific). NGS library preparation, sequencing, and operation of the GUIDE-seq software was performed as previously described except that Needleman-Wunch alignment was incorporated (Tsai et al., 2016).

Target enrichment via rhAmpSeq for multiplexed PCR: To better quantify editing at off-target sites found using GUIDE-seq, multiplex PCR coupled to amplicon NGS was performed using rhPCR (PCR executed in the presence of RNaseH2) (Dobosy et al., 2011) with blocked-cleavable primers. Primers were designed by an algorithm (IDT) for primer cross-comparison and selection based on compatibility with other primers in the multiplex. This amplification technology requires that the primer properly hybridize to a target site before amplification. Mismatches between target and primer prevent unblocking, thereby increasing specificity and eliminating primers dimers. This approach enables efficient production of highly multiplex PCR amplicons in a single tube. For these experiments, gRNA complexes were delivered into HEK293-Cas9 cells as previously described or complexed with Alt-R HiFi Cas9 nuclease v3 to form an active ribonucleoprotein complex (RNP) which was then directly nucleofected into HEK293 cells at 2 μM along with 2 μM Alt-R Cas9 Electroporation Enhancer (IDT). DNA was extracted 48 hrs after electroporation using QuickExtract DNA Extraction Solution (Epicentre). Locus-specific amplification with rh-primers was performed for 10 cycles followed by a 1.5×SPRI bead clean-up. An indexing round of PCR was performed for 18 cycles to incorporate sample-unique P5 and P7 indexes followed by a 1×SPRI bead clean-up and library quantification by qPCR (IDT). PCR amplicons were sequenced on an Illumina MiSeq instrument (v2 chemistry, 150 bp paired end reads) (Illumina, San Diego, Calif., USA). Data were analyzed using a custom-built pipeline. Data were demultiplexed (Picard tools v2.9); forward and reverse reads were merged into extended amplicons (flash v1.2.11) (Magoc et al., 2011); reads were aligned against the GRCh38 genomic reference (bwa mem v0.7.15), and were assigned to targets in the multiplex primer pool (bedtools tags v2.25) (Quinlan et al., 2010). Reads with any base quality score <10 were filtered out. At each target, editing was calculated as the percentage of total reads containing an INDEL within a 10 bp window of the cut site.

Statistics: Two-way Anova test was used to compare quantitative differences (mean±s.d.) between groups; P-values were two-sided and P<0.05 was considered significant. For all bioluminescence experiments, intensity signals were summarized as means±s.d. at baseline and at multiple subsequent time points for each group of mice (Shah et al., 2013). Probabilities of survival were calculated using the Kaplan-Meier method. Statistical tests indicated were performed using Prism software (GraphPad version 7.0c). For the confocal microscopy analysis, data sets were analyzed using unpaired t-tailed test. Data present mean±95% confidence interval. Images were assembled using ImageJ.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., Current Protocols in Molecular Biology, Greene Publishing Associates and John Wiley & Sons, N Y, 1994.
Banerjee et al., J Immunol Methods 355(1-2): 1-13, 2010.
Bennekov et al., Mt. Sinai J. Med. 71 (2): 86-93, 2004.
Bukowski et al., Clinical Cancer Res., 4(10):2337-2347, 1998.
Camacho et al. J Clin Oncology 22(145): Abstract No. 2505 (antibody CP-675206), 2004.
Campbell, Curr. Top. Microbiol. Immunol., 298: 23-57, 2006.
Chothia et al., EMBO J. 7:3745, 1988.
Christodoulides et al., Microbiology, 144(Pt 11):3027-3037, 1998.
Cohen et al. J Immunol. 175:5799-5808, 2005.
Daher et al., Curr Opin Immunol 51: 146-53, 2018.
Davidson et al., J. Immunother., 21(5):389-398, 1998.
Davila et al. PLoS ONE 8(4): e61338, 2013.
Dobosy et al., BMC Biotechnol 11: 80, 2011.
Doulatov et al., Cell Stem Cell. 10:120-36, 2012.
European patent application number EP2537416
Fedorov et al., Sci. Transl. Medicine, 5(215), 2013.
Frolet et al., BMC Microbiol. 10:190 (2010).
Gaj et al., Trends in Biotechnology 31(7), 397-405, 2013.
Gay et al., Clin Lymphoma Myeloma Leuk 17(8): 471-8, 2017.
Hanibuchi et al., Int. J. Cancer, 78(4):480-485, 1998.
Hartmann et al., EMBO Mol Med 9(9): 1183-97, 2017.
Heemskerk et al. Hum Gene Ther. 19:496-510, 2008.
Hellstrand et al., Acta Oncologica, 37(4):347-353, 1998.
Hollander, Front. Immun., 3:3, 2012.
Hubert et al., Proc. Natl. Acad. Sci. USA 96 14523-28, 1999.
Hui and Hashimoto, Infection Immun., 66(11):5329-5336, 1998.
Hurwitz et al. Proc Natl Acad Sci USA 95(17): 10067-10071, 1998.
International Patent Publication No. WO 00/37504
International Patent Publication No. WO 01/14424
International Patent Publication No. WO 2007/069666
International Patent Publication No. WO 2007/069666
International Patent Publication No. WO 98/42752
International Patent Publication No. WO/2014055668
International Patent Publication No. WO1995001994
International Patent Publication No. WO1998042752
International Patent Publication No. WO2000037504
International Patent Publication No. WO200014257
International Patent Publication No. WO2001014424
International Patent Publication No. WO2006/121168
International Patent Publication No. WO2007/103009
International Patent Publication No. WO2009/101611
International Patent Publication No. WO2009/114335
International Patent Publication No. WO2010/027827
International Patent Publication No. WO2011/066342
International Patent Publication No. WO2012/129514
International Patent Publication No. WO2013/071154
International Patent Publication No. WO2013/123061
International Patent Publication No. WO2013/166321
International Patent Publication No. WO2013126726
International Patent Publication No. WO2014/055668
International Patent Publication No. WO2014031687
International Patent Publication No. WO2015016718
International Patent Publication No. WO99/40188
Janeway et al, Immunobiology: The Immune System in Health and Disease, 3^{rd} Ed., Current Biology Publications, p. 433, 1997.
Johnson et al. Blood 114:535-46, 2009.
Jores et al., PNAS U.S.A. 87:9138, 1990.
Kim et al., Genome biology 14(4): R36, 2013.
Kim et al., Nature Biotechnology 31, 251-258, 2013.
Kirchmaier and Sugden, J. Virol., 72(6):4657-4666, 1998.
Kolesnik et al., Methods Mol Biol 967: 235-48, 2013.
Leal, M., Ann N Y Acad Sci 1321, 41-54, 2014.
Lefranc et al., Dev. Comp. Immunol. 27:55, 2003.
Li et al. Nat Biotechnol. 23:349-354, 2005.
Li et al. Proc. Natl. Acad. Sci. USA 89:4275-4279, 1992.
Linnemann, C. et al. Nat Med 21, 81-85, 2015.
Lockey et al., Front. Biosci. 13:5916-27, 2008.
Loewendorf et al., J. Intern. Med. 267(5):483-501, 2010.
Ludwig et al. Nature Biotech., (2):185-187, 2006a.
Ludwig et al. Nature Methods, 3(8):637-646, 2006b.
Magoc Bioinformatics 27(21): 2957-63, 2011.
Marschall et al., Future Microbiol. 4:731-42, 2009.
Mehta et al., Front Immunol 9: 283, 2018.
Mokyr et al. Cancer Res 58:5301-5304, 1998.
Moreno-Mateos et al., Nat Methods 12(10): 982-8, 2015.
Notta et al., Science, 218-221, 2011.
Pardoll, Nat Rev Cancer, 12(4): 252-64, 2012
Parkhurst et al. Clin Cancer Res. 15: 169-180, 2009.
Qin et al., Proc. Natl. Acad. Sci. USA, 95(24):14411-14416, 1998.
Quinlan et al., Bioinformatics 26(6): 841-2, 2010.
Rieder et al., J. Interferon Cytokine Res. (9):499-509, 2009.
Robinson et al., Bioinformatics 26(1): 139-40, 2010.
Rouce et al., Leukemia 30(4): 800-11, 2016.
Rykman, et al., J. Virol. 80(2):710-22, 2006.
Sadelain et al., Cancer Discov. 3(4): 388-398, 2013.
Sambrook et al., Molecular Cloning: A Laboratory Manual, 3^{rd} ed., Cold Spring Harbor Press, Cold Spring Harbor, N.Y. 2001.
Shah et al., PLoS One 8(10): e76781, 2013.
Singh et al., Cancer Research, 68:2961-2971, 2008.
Singh et al., Cancer Research, 71:3516-3527, 2011.
Takahashi et al., Cell, 126(4):663-76, 2007.
Terakura et al. Blood. 1:72-82, 2012.
Tsai et al., Nat Biotechnol 33(2): 187-97, 2015.
Tsai et al., Nat Biotechnol 34(5): 483, 2016.
Turtle et al., Curr. Opin. Immunol., 24(5): 633-39, 2012.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 5,994,136
U.S. Pat. No. 6,013,516
U.S. Pat. No. 6,103,470
U.S. Pat. No. 6,207,156
U.S. Pat. No. 6,225,042
U.S. Pat. No. 6,355,479
U.S. Pat. No. 6,362,001
U.S. Pat. No. 6,410,319
U.S. Pat. No. 6,416,998
U.S. Pat. No. 6,544,518
U.S. Pat. No. 6,790,662
U.S. Pat. No. 7,109,304
U.S. Pat. No. 7,442,548
U.S. Pat. No. 7,446,190
U.S. Pat. No. 7,598,364
U.S. Pat. No. 7,989,425
U.S. Pat. No. 8,008,449
U.S. Pat. No. 8,017,114
U.S. Pat. No. 8,058,065
U.S. Pat. No. 8,071,369
U.S. Pat. No. 8,119,129
U.S. Pat. No. 8,129,187
U.S. Pat. No. 8,183,038
U.S. Pat. No. 8,268,620
U.S. Pat. No. 8,329,867
U.S. Pat. No. 8,354,509
U.S. Pat. No. 8,546,140
U.S. Pat. No. 8,691,574
U.S. Pat. No. 8,735,553
U.S. Pat. No. 8,741,648
U.S. Pat. No. 8,900,871
U.S. Pat. No. 9,175,268
U.S. Patent Publication No. 2010/0210014
U.S. patent Ser. No. 12/478,154
U.S. Patent Publication No. 2002131960
U.S. Patent Publication No. 2003/0211603
U.S. Patent Publication No. 2005/0260186
U.S. Patent Publication No. 2006/0104968
U.S. Patent Publication No. 2009/0004142
U.S. Patent Publication No. 2009/0017000
U.S. Patent Publication No. 2009/0246875
U.S. Patent Publication No. 2011/0104125
U.S. Patent Publication No. 2011/0301073
U.S. Patent Publication No. 20110008369
U.S. Patent Publication No. 2012/0276636
U.S. Patent Publication No. 2013/0315884
U.S. Patent Publication No. 20130149337
U.S. Patent Publication No. 2013287748
U.S. Patent Publication No. 2014/0120622
U.S. Patent Publication No. 2014022021
U.S. Patent Publication No. 20140294898
Van Gassen et al., Cytometry 87(7): 636-45, 2015.
Varela-Rohena et al. Nat Med. 14: 1390-1395, 2008.
Vera et al., Blood 108(12): 3890-7, 2006.
Wang et al. J Immunother. 35(9):689-701, 2012.
Wu et al., Adv. Cancer Res., 90: 127-56, 2003.
Wu et al., Cancer, 18(2): 160-75, 2012.
Yamanaka et al., Cell, 131(5):861-72, 2007.
Yu et al., Science, 318:1917-1920, 2007.
Zysk et al., Infect. Immun. 68(6):3740-43, 2000.

What is claimed is:

1. An isolated natural killer (NK) cell engineered to express (1) a chimeric antigen receptor (CAR) and/or a T cell receptor (TCR) and (2) human IL-15 (hIL-15) and to have essentially no expression of CISH.

2. The NK cell of claim 1, wherein the NK cell is engineered to express a CAR.

3. The NK cell of claim 1, wherein the NK cell is engineered to express a TCR.

4. The NK cell of claim 1, wherein the NK cell is engineered to express a CAR and TCR.

5. The NK cell of claim 1, wherein the NK cell is derived from cord blood, peripheral blood, bone marrow, CD34⁺ cells, or iPSCs.

6. The NK cell of claim 1, wherein the NK cell is derived from cord blood.

7. The NK cell of claim 6, wherein the cord blood has previously been frozen.

8. The NK cell of claim 1, wherein the CAR and/or TCR has antigenic specificity for CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD33, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2.

9. The NK cell of claim 1, wherein the CAR has antigenic specificity for CD19.

10. The method of claim 1, wherein the NK cell further expresses a second cytokine.

11. The method of claim 10, wherein the cytokine is IL-21 or IL-12.

12. The method of any of claims 1-11, wherein the NK cell has activated mammalian target of rapamycin (mTOR) signaling.

13. The method of claim 12, wherein the NK cell has increased JAK/STAT signaling.

14. A method for producing NK cells of any one of claims 1-14 comprising:
(a) obtaining a starting population of NK cells;
(b) culturing the starting population of NK cells in the presence of artificial presenting cells (APCs);
(c) introducing a CAR and/or TCR expression vector into the NK cells;
(d) expanding the NK cells in the presence of APCs, thereby obtaining expanded NK cells; and
(e) disrupting the expression of CISH in the expanded NK cells.

15. The method of claim 14, wherein disrupting expression comprises using CRISPR-mediated gene silencing.

16. The method of claim 15, wherein CRISPR-mediated gene silencing comprises contacting the CAR NK cells with sgRNA and Cas9.

17. The method of claim 11, wherein the sgRNA targets exon 4 of CISH.

18. The method of claim 17, wherein the sgRNA comprises SEQ ID NOs:1-2.

19. The method of claim 14, wherein the starting population of NK cells is obtained by isolating mononuclear cells using a ficoll-paque density gradient.

20. The method of claim 14, wherein the APCs are gamma-irradiated APCs.

21. The method of claim 14, wherein the APCs are universal APCs (uAPCs).

22. The method of claim 14, wherein the APCs are engineered to express 41BB and IL-21.

23. The method of claim 21, wherein the uAPCs are engineered to express (1) CD48 and/or CS1 (CD319), (2) membrane-bound interleukin-21 (mbIL-21), and (3) 41BB ligand (41BBL).

24. The method of claim 14, wherein the NK cells and APCs are present at a 1:2 ratio.

25. The method of claim 14, wherein the NK cells of step (b) and (d) are further expanded in the presence of IL-2.

26. The method of claim 25, wherein the IL-2 is present at a concentration of 100-300 U/mL.

27. The method of claim 25, wherein IL-2 is present at a concentration of 200 U/mL.

28. The method of claim 14, wherein introducing comprises transduction.

29. The method of claim 14, wherein the CAR and/or TCR expression construct is a lentiviral vector or retroviral vector.

30. The method of claim 14, wherein the NK cells are GMP-compliant.

31. The method of claim 14, wherein the NK cells are allogeneic.

32. The method of claim 14, wherein the NK cells are autologous.

33. The method of claim 14, wherein the CAR and/or TCR has antigenic specificity for CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2.

34. The method of claim 14, wherein the CAR has antigenic specificity for CD19.

35. The method of claim 14, wherein the CAR and/or TCR expression construct further expresses a cytokine.

36. The method of claim 35, wherein the cytokine is IL-15, IL-21, or IL-12.

37. The method of claim 14, further comprising cryopreserving the population of expanded NK cells.

38. A pharmaceutical composition comprising a population of NK cells of any one of claims 1-11 or NK cells produced by the method of any one of claims 14-37 and a pharmaceutically acceptable carrier.

39. A composition comprising an effective amount of NK cells of any one of claims 1-11 or NK cells produced by the method of any one of claims 14-37 for use in the treatment of a disease or disorder in a subject.

40. The use of a composition comprising an effective amount of NK cells of any one of claims 1-11 or NK cells produced by the method of any one of claims 14-37 for the treatment of an immune-related disorder in a subject.

41. A method of treating an immune-related disorder in a subject comprising administering an effective amount of NK cells of any one of claims 1-11 or NK cells produced by the method of any one of claims 14-37 to the subject.

42. The method of claim 41, wherein the immune-related disorder is a cancer, autoimmune disorder, graft versus host disease, allograft rejection, or inflammatory condition.

43. The method of claim 41, wherein the immune-related disorder is an inflammatory condition and the immune cells have essentially no expression of glucocorticoid receptor.

44. The method of claim 43, wherein the subject has been or is being administered a steroid therapy.

45. The method of claim 41, wherein the NK cells are autologous.

46. The method of claim 41, wherein the NK cells are allogeneic.

47. The method of claim 41, wherein the immune-related disorder is a cancer.

48. The method of claim 47, wherein the cancer is a solid cancer or a hematologic malignancy.

49. The method of claim 41, further comprising administering at least a second therapeutic agent.

50. The method of claim 49, wherein the at least a second therapeutic agent comprises chemotherapy, immunotherapy, surgery, radiotherapy, or biotherapy.

51. The method of claim 49, wherein the NK cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

52. The method of claim 49, wherein the NK cells with essentially no expression of CISH have enhanced function as compared to NK cells with expression of CISH.

53. The method of claim 52, wherein the enhanced function is measured by intracellular staining for IFN-γ and TNF-α, CD107a degranulation, and tumor killing by ⁵¹Cr release assay.

54. The method of claim 52, wherein the enhanced function is measured by increased expression of granzyme-b, perforin, TRAIL, CD3z, Eomes, T-bet, DAP12, DNAM, CD25 and/or Ki67.
